(19) United States
(12) Patent Application Publication
(10) Pub. No.: US 2025/0195273 A1
(43) Pub. Date: Jun. 19, 2025

(54) AN IMPLANT, A DEPLOYER TOOL FOR INSERTING AN IMPLANT, AND A SYSTEM FOR FACILITATING THE CONDUCTION OF FLUID

(71) Applicant: Agency for Science, Technology and Research, Singapore (SG)

(72) Inventors: Tao Yi David CHEN, Singapore (SG); Jia Yun HEE, Singapore (SG); Shihan Bryan HO, Singapore (SG); Preeti MOHAN, Singapore (SG)

(21) Appl. No.: 18/851,867

(22) PCT Filed: Mar. 30, 2022
(86) PCT No.: PCT/SG2022/050178

§ 371 Date: Sep. 27, 2024

(51) Int. Cl. A61F 9/007; A61B 17/34; A61M 25/00

(52) U.S. Cl. CPC A61F 9/00781; A61B 17/3468; A61M 25/007

(57) ABSTRACT

There is provided an implant configured to be deployed in an eye by a deployer tool to facilitate conduction of fluid from a posterior chamber to a suprachoroidal space of the eye, the implant comprising, an elongate member having a proximal segment configured to be disposed in the posterior chamber of the eye and a distal segment configured to be disposed in the suprachoroidal space of the eye, wherein the elongate member is configured to maintain a bent configuration that substantially complies with anatomical curvatures of the posterior chamber and the suprachoroidal space, when deployed in the eye. There is also provided a deployer tool for inserting an implant as disclosed herein into an eye to facilitate conduction of fluid from a posterior chamber to a suprachoroidal space of the eye, and a system for facilitating the conduction of fluid from a posterior chamber to a suprachoroidal space of an eye.

### TECHNICAL FIELD

The present disclosure relates broadly to an implant, a deployer tool for inserting an implant, and a system for facilitating the conduction of fluid.

### BACKGROUND

Glaucoma is a group of diseases resulting in damage to the optic nerve due to abnormally high pressures in the eye. The two most common types of clinically characterized glaucoma in humans include primary open-angle glaucoma (POAG) and primary angle closure glaucoma (PACG). Both types of glaucoma are characterized by progressive and irreversible destruction of optic nerve axons and degeneration of the retinal ganglion cells, frequently in association with increased intraocular pressure (IOP).

Although the prevalence of POAG is higher than PACG globally, PACG is a disproportionately larger problem in East Asia. PACG is a more aggressive form of glaucoma and accounts for about 90 percent of all cases of glaucoma-related blindness in China.

In general, IOP has a normal range between about 10-21 mmHg. It is regulated by a balance between secretion of aqueous humour (AH) by the ciliary body in the posterior chamber, and drainage of AH from the anterior chamber angle, either through the trabecular meshwork/Schlemm's canal (TM/SC) or via the uveoscleral outflow pathway through the iris root. These are also known as the conventional and unconventional pathways respectively.

In ACG, the peripheral iris is in contact with the TM and peripheral cornea, blocking egress of AH via the outflow pathways. Iridocorneal contact is due to forward bulging of the peripheral iris, due to a pressure difference between posterior and anterior chambers. This may arise due to increased flow resistance for AH through the slit between the iris and lens in association with anatomical abnormalities, abnormalities in iris thickness and position, or plateau iris configuration.

In chronic ACG, there is a gradual closure of the chamber angle described above. Peripheral anterior synechiae (PAS; adhesions of the peripheral iris to structures in the angle of the anterior chamber) form as a result of prolonged appositional contact between the iris and the trabecular meshwork. Such prolonged obstruction eventually leads to irreversible changes inside the meshwork, which permanently block the conventional outflow pathway at the site of the PAS.

The main aim of treatment of chronic ACG is to eliminate the underlying pathophysiological mechanism causing the angle closure-mainly pupil block and peripheral angle crowding/plateau iris. Apart from eye drops, laser peripheral iridotomy (LPI) is usually the first line of treatment, however its role as long-term therapy is unsatisfactory, as up to about 53% of patients would eventually require surgery due to poorly controlled IOP and failed medical therapy. Laser iridoplasty is further indicated if the angle remains occludable after laser peripheral iridotomy, but this procedure is ineffective in the presence of established, extensive PAS.

Lens extraction is increasingly being seen as an appropriate first-line treatment compared to laser peripheral iridotomy in ACG, due to its increased efficacy and more cost-effectiveness based on the EAGLE (Effectiveness in Angle-Closure Glaucoma of Lens Extraction)trial. Unfortunately, lens extraction alone may not eliminate residual PAS, especially in patients with chronic ACG.

The final treatment option for such patients with chronic ACG refractory to medications or the above treatments is trabeculectomy, or the insertion of glaucoma drainage devices (GDD) e.g., tube-based Molteno, Baerveldt or Ahmed implants. Trabeculectomy is a technically complex procedure that can result in failure due to scarring, decreased quality of life due to bleb-related foreign body sensation, induced astigmatism and secondary cataracts. Rates of reoperation are also relatively high.

As such, there exists a treatment gap for a safer and effective modality to better improve treatment outcomes for patients with chronic ACG, especially for patients with moderate severity of disease. As an alternative to traditional glaucoma treatments, minimally invasive glaucoma surgery (MIGS) has shown promise for the future management of glaucoma. The features of MIGS have been proposed as: (i) ab interno, microincisional approach; (ii) minimal trauma/disruption to normal anatomy and physiology; (iii) demonstrable/reliable IOP lowering; (iv) extremely high safety profile; and (v) rapid post-op recovery, with minimal need for follow-up.

Currently available MIGS approaches have been designed for the treatment of mild to moderate POAG. Attempts at adapting these existing devices for chronic ACG have not been successful, with failure rates between about 30-40% (as compared to failure rates of less than about 5%, when used in patients in POAG). It is recognised that developing a MIGS-like approach to chronic ACG will require the design, development and testing of a new device, for a stronger focus and eventual implementation in an Asian patient population.

Existing MIGS devices are unsuitable for treatment of chronic angle closure glaucoma due to the following factors: (i) inability to achieve adequate visualisation and surgical access to the anterior chamber angle for the deployment of the MIGS device; (ii) dysfunction of conventional outflow pathways (e.g. trabecular meshwork/Schlemm Canal (TM/SC)) in patients with chronic angle closure, especially with established PAS of more than 6 months duration; (iii) risk of iris occlusion or re-formation of peripheral anterior synechiae enveloping the proximal inflow of the MIGS device, leading to failure of drainage; (iv) risk of prolonged and persistent endothelial cell loss due to anterior chamber location of the MIGS device, which may be exacerbated in patients with chronic angle closure (this risk is particularly relevant with regards to future regulatory approval of this device, due to the withdrawal of the Alcon Cypass system in August 2018); and (v) increased risk of scarring/fibrosis for subconjunctival outflow MIGS approaches, leading to repeated salvage procedures i.e. needling or subsequent device failure.

Thus, there is a need for an implant, a deployer tool for inserting an implant, and a system for facilitating the conduction of fluid, which seeks to address or at least ameliorate one of the above problems.

### SUMMARY

In one aspect, there is provided an implant configured to be deployed in an eye by a deployer tool to facilitate conduction of fluid from a posterior chamber to a suprachoroidal space of the eye, the implant comprising, an elongate member having a proximal segment configured to be disposed in the posterior chamber of the eye and a distal segment configured to be disposed in the suprachoroidal space of the eye, wherein the elongate member is configured to maintain a bent configuration that substantially complies with anatomical curvatures of the posterior chamber and the suprachoroidal space, when deployed in the eye.

In one embodiment, the elongate member is configured to be straightened to facilitate loading of the implant into the deployer tool for deployment; and to return to the bent configuration when deployed in the eye.

In one embodiment, the elongate member is pre-formed in the bent configuration with a bend angle that substantially matches an anatomical angle located between the posterior chamber and the suprachoroidal space of the eye.

In one embodiment, the elongate member has a bending stiffness of no more than 0.5 N.mm², such that the elongate member is able to move along a delivery path formed by an access needle of the deployer tool from a sulcus or a portion of the ciliary body to the suprachoroidal space of the eye, without substantially deforming the access needle.

In one embodiment, the distal segment of the elongate member is curved with a radius of curvature that is substantially identical to a radius of curvature of an inner sclera of the eye.

In one embodiment, the elongate member is bevelled at its proximal tip such that the bevelled surface is configured to face away from a posterior surface of an iris, when the implant is deployed in the eye.

In one aspect, there is provided a deployer tool for inserting an implant into an eye to facilitate conduction of fluid from a posterior chamber to a suprachoroidal space of the eye, the deployer tool comprising, a guide tube for accessing the posterior chamber of the eye through an insertion site, said guide tube comprising a distal tip that is configured to abut a ciliary sulcus or a portion of a ciliary body of the eye; and an access needle configured to be disposed within the guide tube and to create a delivery path from the sulcus or a portion of the ciliary body to the suprachoroidal space of the eye; wherein the deployer tool allows for movement of the implant in relation to the access needle along the delivery path such that a distal segment of the implant is disposed in the suprachoroidal space of the eye, and a proximal segment of the implant is disposed in the posterior chamber of the eye.

In one embodiment, the deployer tool further comprises an implant actuator configured to detachably engage to the implant and configured to move the implant distally in relation to the access needle along the delivery path.

In one embodiment, the access needle comprises a distal tip configured to dissect tissue so as to create the delivery path; wherein the delivery path comprises a first section extending from the sulcus or a portion of the ciliary body to an inner surface of a sclera and a second section extending from the inner surface of the sclera to the suprachoroidal space; and wherein the first section and second section form a bent angle along the delivery path.

In one embodiment, the access needle is pre-formed in a bent configuration and configured to be straightened when disposed within the guide tube and to return to the bent configuration when extended from the distal tip of the guide tube for the creation of the delivery path.

In one embodiment, the access needle has a stiffness of no more than 2.5 N/mm to facilitate bending of the access needle at the bent angle along the delivery path.

In one embodiment, the guide tube has a Young's modulus of at least 7.5 GPa such that the guide tube does not substantially deflect when abutting the ciliary sulcus or a portion of the ciliary body of the eye.

In one embodiment, the deployer tool further comprises an access needle holder, an implant actuator holder and a guide tube holder disposed thereon, wherein the access needle holder is configured to engage a proximal end of the access needle, the implant actuator holder is configured to engage the implant actuator and the guide tube holder is configured to engage the guide tube, and optionally wherein the access needle holder, implant actuator holder and/or the guide tube holder are each further coupled to a respective control configured to actuate the access needle, implant actuator and optionally, the guide tube. In one aspect, there is provided a system for facilitating the conduction of fluid from a posterior chamber to a suprachoroidal space of an eye, the system comprising, an implant comprising, an elongate member having a proximal segment and a distal segment, wherein the elongate member is configured to maintain a bent configuration that substantially complies with anatomical curvatures of the posterior chamber and the suprachoroidal space, when deployed in the eye; and a deployer tool comprising, a guide tube for accessing the posterior chamber of the eye through an insertion site, said guide tube comprising a distal tip that is configured to abut a ciliary sulcus or a portion of a ciliary body of the eye; and an access needle configured to be disposed within the guide tube and to create a delivery path from the sulcus or a portion of the ciliary body to the suprachoroidal space of the eye;

wherein the deployer tool allows for movement of the implant in relation to the access needle in a distal direction along the delivery path such that a distal segment of the implant is disposed in the suprachoroidal space of the eye, and a proximal segment of the implant is disposed in the posterior chamber of the eye.

In one embodiment, the elongate member is configured to be straightened to facilitate loading of the implant on a distal end of the access needle. In one embodiment, the implant and access needle are configured to be in a retracted position and disposed within the guide tube prior to deployment.

In one embodiment, the insertion site is located on a cornea of the eye; wherein the distal tip of the guide tube is configured to be inserted through the corneal insertion site into an anterior chamber of the eye, and to be inserted across a pupil of the eye, and to be moved along a posterior surface of an iris to abut the ciliary sulcus or a portion of the ciliary body of the eye.

In one embodiment, the deployer tool further comprises, an access needle holder configured to engage a proximal end of the access needle and configured to extend the access needle distally from the distal tip of the guide tube to allow a distal tip of the access needle to dissect tissue and create the delivery path; wherein the delivery path comprises a first section extending from the sulcus or a portion of the ciliary body to an inner surface of a sclera and a second section extending from the inner surface of the sclera to the suprachoroidal space; and wherein the first section and second section form a bent angle along the delivery path.

In one embodiment, the deployer tool further comprises, an implant actuator configured to detachably engage the implant; and an implant actuator holder configured to engage the implant actuator and configured to move the implant actuator such that the implant actuator engages the implant and the implant is moved distally in relation to the access needle along the delivery path.

In one embodiment, the access needle holder is further configured to retract the access needle proximally such that the distal tip of the access needle is more proximal in relation to the distal tip of the implant actuator.

### Definitions

Various parts of the deployer tool and implant as disclosed herein will be described using the terms “proximal” and “distal”. The term “proximal” as defined herein is to be interpreted broadly to mean in the direction of an operator, e.g., a medical professional. The term “distal” as defined herein is to be interpreted broadly to mean in the direction of a subject, e.g., a patient. When referring to specific feature(s) of the implant, the terms “proximal” and “distal” refer to the relative positions of the feature(s), e.g., toward the operator or toward the subject, when the implant is inserted in the subject.

The terms “anterior” and “posterior” as used herein are to be interpreted in accordance with their generally understood anatomical interpretation. Thus, “anterior” refers to a front of a subject e.g., human, and “posterior” refers to a rear of the subject.

The terms “medial” and “lateral” as used herein are to be interpreted in accordance with their generally understood anatomical interpretation. Thus, “medial” refers to a direction towards a midline of a subject e.g., human, and “lateral” refers to a direction away from the midline of the subject.

The term “biocompatible” as used herein is to be interpreted broadly to refer to the ability of an implant to perform its intended function without substantially producing any undesirable local or systemic effects in a subject receiving the implant.

The term “bioinert” as used herein is to be interpreted broadly to refer to a material that does not substantially elicit an immune response from a human or animal when it is disposed within an in-vivo biological environment.

The term “implant” as used herein is to be interpreted broadly to refer to any object that is intended for placement in the body of a mammal, such as a human, that is not a living tissue.

The terms “eye” and “eyeball” as interchangeably used herein refer to a human eye or mammalian eye.

The term “anterior chamber” as used herein refers to a region/space of the eye that is behind the cornea and in front of the iris.

The term “posterior chamber” as used herein refers to a region/space of the eye that is behind the iris and in front of the lens.

The term “suprachoroidal space” as used herein refers to a space of an eye that is between the choroid and the sclera that traverses the circumference of the posterior segment of the eye.

The term “treating” as used herein is to be interpreted broadly to mean attempting to inhibit/reduce the progression of a disease (e.g., glaucoma) temporarily or attempting to stop the progression of the disease permanently. The disease may not need to be effectively treated eventually.

The term “substrate” as used herein is to be interpreted broadly to refer to any supporting structure.

The term “layer” when used to describe a first material is to be interpreted broadly to refer to a first depth of the first material that is distinguishable from a second depth of a second material. The first material of the layer may be present as a continuous film, as discontinuous structures or as a mixture of both. The layer may also be of a substantially uniform depth throughout or varying depths. Accordingly, when the layer is formed by individual structures, the dimensions of each of individual structure may be different. The first material and the second material may be same or different and the first depth and second depth may be same or different.

The term “substantially transparent to light” when used herein to describe an object is to be interpreted broadly to mean that 50% or more of the incident light normal to surface of the object can be transmitted through the object. In some examples, the object that is substantially transparent to light allow 60% or more, 65% or more, 70% or more, 80% or more, 85% or more, 90% or more or 95% or more of the incident light normal to surface of the object to be transmitted. In one example, the object that is substantially transparent to light allow above 70% of the incident light normal to surface of the object to be transmitted.

The term “micro” as used herein is to be interpreted broadly to include dimensions from about 1 micron to about 1000 microns.

The term “nano” as used herein is to be interpreted broadly to include dimensions less than about 1000 nm.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. It is to be appreciated that the individual numerical values within the range also include integers, fractions and decimals. Furthermore, whenever a range has been described, it is also intended that the range covers and teaches values of up to 2 additional decimal places or significant figures (where appropriate) from the shown numerical end points. For example, a description of a range of 1% to 5% is intended to have specifically disclosed the ranges 1.00% to 5.00% and also 1.0% to 5.0% and all their intermediate values (such as 1.01%, 1.02% . . . 4.98%, 4.99%, 5.00% and 1.1%, 1.2% . . . 4.8%, 4.9%, 5.0% etc.,) spanning the ranges. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Non-limiting embodiments of an implant configured to be deployed in an eye/eyeball by a deployer tool to facilitate conduction of fluid from a posterior chamber to a suprachoroidal space of the eye, a deployer tool for inserting an implant into an eye to facilitate conduction of fluid from a posterior chamber to a suprachoroidal space of the eye, a system e.g., delivery system for facilitating the conduction of fluid from a posterior chamber to a suprachoroidal space of an eye, and a method of inserting an implant into an eye to facilitate conduction of fluid from a posterior chamber to a suprachoroidal space of the eye, are disclosed hereinafter.

Various embodiments of the implant, deployer tool, system and method as disclosed herein may be used for the treatment of eye diseases e.g., chronic angle closure glaucoma. Various embodiments of the implant, deployer tool, system and method as disclosed herein may be used for the surgical treatment of patients with chronic angle closure glaucoma, used in conjunction with the current standard of care (e.g., lens extraction or phacoemulsification).

In particular, various embodiments of the implant, deployer tool, system and method as disclosed herein may provide a delivery system that uses a minimally-invasive procedure, which circumvent issues associated with existing MIGS devices and issues which are relevant to chronic angle closure glaucoma by offering one or more of the following advantages: (i) circumventing issues of visualization and deployment in narrow iridocorneal angles; (ii) augmenting the unconventional (uveoscleral) outflow pathway, which is less prone to dysfunction compared to the conventional outflow pathway; (iii) bypassing any re-formed peripheral anterior synechiae that would predispose anterior chamber devices for occlusion and subsequent failure; (iv) reducing the risk for chronic endothelial cell loss compared to other approaches with implants retained in the anterior chamber; and (v) incurring less scarring or fibrosis compared to ab-externo or subconjunctival outflow approaches.

### Implant

In various embodiments, there is provided an implant configured to be deployed in an eye by a deployer tool to facilitate conduction of fluid from a posterior chamber to a suprachoroidal space of the eye, the implant comprising, an elongate member having a proximal segment configured to be disposed in the posterior chamber of the eye and a distal segment configured to be disposed in the suprachoroidal space of the eye, wherein the elongate member is configured to maintain a bent configuration that substantially complies with anatomical curvatures of the posterior chamber and the suprachoroidal space, when deployed in the eye.

In various embodiments, the implant functions to create and maintain an outflow drainage shunt/tract/pathway/passageway from the posterior chamber of the eye to the suprachoroidal space, for drainage of fluid e.g., aqueous humour, hence augmenting the physiologic uveoscleral outflow pathway. In one embodiment, the implant is an ocular implant. In one embodiment, the implant is a drainage shunt implant for draining aqueous humour.

In various embodiments, the elongate member comprises an inlet port disposed at a proximal end/tip of the elongate member, said inlet port being in fluid communication with, and being configured to, receive fluid from the posterior chamber of the eye. In various embodiments, the elongate member further comprises an outlet port disposed at a distal end/tip of the elongate member, said outlet being in fluid communication with, and being configured to, discharge fluid into the suprachoroidal chamber. In various embodiments, the elongate member further comprises a passageway, said passageway being configured to convey fluid from the inlet port to the outlet port. The passageway may include but is not limited to a single lumen disposed within a hollow tube, multiple interconnected or non-interconnected lumens e.g., multiple parallel lumens disposed within a hollow tube, an open channel with various cross-sectional profile such as a semi-circular cross-sectional profile, a plurality of interconnected chambers disposed between the inlet and outlet ports. In one embodiment, the implant takes the form of a hollow elongate member.

In various embodiments, the implant is configured to maintain a bent configuration that substantially complies with anatomical curvatures of the posterior chamber and the suprachoroidal space, when deployed/implanted in the eye. In various embodiments, the implant may be bent such that the proximal segment of the elongate member is spaced apart from a posterior surface of an iris of the eye, when deployed in the eye. In other words, the implant may be bent such that the proximal segment of the elongate member does not abut on the posterior surface of the iris This may advantageously minimise or prevent the implant from rubbing or scratching against the iris, which may cause irritation or inflammation of the iris tissue, and wear and tear to the iris tissue and/or the implant.

In various embodiments, the implant is configured to be straightened to facilitate loading of the implant into the deployer tool for deployment, and to return to the bent configuration when deployed in the eye. In various embodiments, the implant may be configured to be biased in the bent configuration, such that the implant is able to return to the bent configuration after being deformed e.g., straightened. During deployment, the implant may be straightened as it traverses a delivery path comprising straight and bent paths before implantation. The implant may be sufficiently flexible such that it retains its bent form despite this straightening (bending stress being less than yield strength). The implant may possess creep resistance such that the implant is capable of retaining the bent configuration despite being straightened for a period of time, e.g., for the duration of deployment. The implant may be configured to return to the bent configuration after being maintained in a substantially straightened state for a period of up to about 5 minutes, up to about 10 minutes, up to about 15 minutes, up to about 20 minutes, up to about 25 minutes, or up to about 30 minutes. For example, the implant may be made of polytetrafluoroethylene (PTFE) with an inner diameter of from about 0.1 mm to about 0.8 mm.

In various embodiments, the implant is pre-formed/pre-bent in the bent configuration with a bend angle that substantially matches, mimics or approximates an anatomical angle located between the posterior chamber and the suprachoroidal space of the eye. In various embodiments, the implant may be bent at an angle or profile that is substantially identical or similar to that found at an interface/interfacial region between the posterior chamber and suprachoroidal space. The anatomical angle located between the posterior chamber and the suprachoroidal space of the eye may be defined by a first section extending in a substantially medial or lateral direction from a ciliary sulcus or a portion of a ciliary body in the posterior chamber to an inner surface of a sclera of the eye; and a second section extending in a substantially posterior direction from the inner surface of the sclera into the suprachoroidal space. The angle formed by the first section and second section may be defined as the anatomical angle located between the posterior chamber and the suprachoroidal space of the eye.

In various embodiments, the bent configuration may comprise a bend angle ranging from about 30° to about 60°. The bend angle is defined as the included angle between a first imaginary line drawn from and perpendicular to the proximal segment and a second imaginary line drawn from and perpendicular to the distal segment of the elongate member. The bend angle may be in a range with start and end points selected from the following group of numbers: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60°. The bent configuration may comprise a bend radius ranging from about 0.5 mm to about 1 mm. The bend radius may be in a range with start and end points selected from the following group of numbers: 0.5, 0.55, 0.6, 0.65, 0.7, 0.75, 0.8, 0.85, 0.9, 0.95, and 1 mm. For example, the elongate member may be bendable at one or more locations on the proximal segment and/or one or more locations on the distal segment. The elongate member may comprise one or more bendable segment e.g., a joint, between the proximal segment and distal segment that permits bending/flexing.

In various embodiments, the implant may be made of materials that facilitate a bent configuration/state. The implant may be made of materials that possess one or more mechanical properties that facilitate a bent configuration/state. The implant may be made of materials that possess one or more mechanical properties that simulate one or more mechanical properties of a part of an eye, e.g., an iris. The one or more mechanical properties may include but are not limited to creep, ductility, elasticity, flexibility, plasticity, and stiffness. The implant or a part thereof may be made of materials that are substantially flexible/bendable/pliable. The implant may be sufficiently flexible such that the implant substantially conforms or yields in response to one or more forces applied by surrounding tissue on the implant. For example, the implant may be configured to bend when pressed down in a posterior direction by the iris. The implant may have a Young's modulus that simulates the Young's modulus of a human iris. The Young's modulus of the implant may be lower than, similar to, or higher than the Young's modulus of the human iris. For example, an implant having a Young's modulus that simulates the Young's modulus of the human iris may be achieved by a combination of one or more of the following: (1) a material having a Young's modulus less than 50 Mpa, such as a polymer (e.g. poly(Styrene-block-IsoButylene-block-Styrene) (SIBS)), and (2) a joint that permits flexing. In various embodiments, the implant may be made of one or more materials that are substantially transparent to light so that the implant does not impede vision of a subject when deployed in the eye.

In various embodiments, the implant is configured to be substantially flexible. The implant or its distal segment may have a bending stiffness that is sufficiently low such that the implant or its distal segment is flexible enough to pass through a bent path (e.g., angle between the posterior chamber and suprachoroidal) during deployment. In various embodiments, the implant or its distal segment may have a bending stiffness of less than about 0.5 N.mm², less than about 0.4 N.mm², less than about 0.3 N.mm², less than about 0.2 N.mm², or less than about 0.1 N.mm². In various embodiments, the implant may have a Young's modulus of more than about 20 GPa, more than about 25 GPa, more than about 30 GPa, more than about 35 GPa, more than about 40 GPa, more than about 45 GPa, or more than about 50 GPa.

For example, the implant or its distal segment may have a bending stiffness of less than about 0.5 N.mm². The implant may also be configured to be substantially more flexible than an access needle for guiding the implant, such that the implant does not deform (e.g., straighten) the guiding access needle when passing over it. In various embodiments, the implant may have a bending stiffness that is lower than that of the access needle. In various embodiments, the lower bending stiffness of the implant allows the implant to follow a curvature or a bent shape of the access needle. In one embodiment, the elongate member has a bending stiffness of no more than about 0.5 N.mm², such that the elongate member is able to move along a delivery path formed by an access needle of the deployer tool from a sulcus or a portion of a ciliary body to the suprachoroidal space of the eye, without substantially deforming the access needle e.g., when passing over the access needle. The implant may have a Young's modulus of more than about 20 GPa, such that it has sufficient column strength to withstand buckling during deployment. The implant may possess sufficient creep resistance, such that after being straightened for a duration of about 10 minutes, the change of its bend angle is less than about 20%.

In various embodiments, the implant may be specially shaped to facilitate a bent configuration/state.

In various embodiments, the implant may comprise cutouts (i.e., removal of material) at the proximal segment and/or distal segment of the elongate member, such that a cross-sectional area of the elongate member at the proximal segment and/or distal segment is reduced, thereby reducing a second moment of area and hence lowering a bending strength of the implant at the proximal segment and/or distal segment. For example, the cutout may comprise removal of a semi-cylindrical portion of material at the proximal segment of the elongate member, such that the proximal segment of the elongate member comprises a semi-cylindrical shape/profile.

In various embodiments, the implant may also comprise cutouts at a portion of the elongate member between the proximal segment and distal segment, to facilitate bending of the elongate member. The cutouts may have different shapes and patterns and may include but are not limited to triangular, square, rectangular, polygonal, pentagonal, hexagonal, octagonal, circular, elliptical, semi-circular shapes, coiled shapes, zig-zag shapes, sinusoidal shapes, spaced-lines, and the like. For example, the cutout may involve removal of material in a spiral manner, creating a “spring” configuration at the portion of the elongate member between the proximal segment and distal segment. The cutouts may be achieved by laser cutting e.g., laser cutting a spiral in polyimide or nitinol (a metal alloy of nickel and titanium) material.

In various embodiments, the portion of the elongate member between the proximal segment and distal segment may be configured in a flexible shape that facilitates bending. For example, the portion of the elongate member between the proximal segment and distal segment may be pleated to facilitate bending. For example, the implant may comprise a plurality of struts e.g., thin struts which confer a tubular shape while maintaining flexibility. The struts may, for example, take the shape of a coil, or modular rings connected by longitudinal segments. As an example, a bent portion of the implant may have a flexible bellows-like or accordion-like structure, with alternating rings of a first diameter and a second diameter formed of a rigid material such as nitinol, wherein the first and second diameters are different.

In various embodiments, the implant has dimensions (e.g., length, thickness, diameter and circumference) that are suitably sized in relation to ocular dimensions of an adult human eye for a normal and a diseased state, for the purposes of creating a drainage shunt between the posterior chamber of the eye and the suprachoroidal space of the eye.

For example, as described in one study (Janakiranman Palani, “Change of ocular dimensions in different types of glaucoma”, Visual Science Academy, available online on 1 Feb. 2021), ocular dimensional parameters considered in glaucoma may include axial length, keratometry, anterior chamber depth, anterior chamber volume, lens thickness, pachymetry, relative lens position and len-axial length factor. Ocular dimension changes with different subtypes of glaucoma, starting with the axial length, which is found to be shorter in occludable angles and primary angle-closure glaucoma ranging from 21.62 mm to 22.83 mm as compared to other types of glaucoma. Corneal curvature measured with keratometry shows a steeper ‘K’ value ranging from 43.75D to 44.87D in the case of angle-closure glaucoma than other types of glaucoma. The anterior chamber depth is found to be shallow in all types of ACG ranging from 1.84 mm to 2.69 mm. Anterior chamber volume which is a unique factor found to be less than 100 μl in case of ACG except for plateau iris configuration (i.e., in 91 μl in extreme narrow angles and 113 μl in case of plateau iris configuration). The central corneal thickness was found to be thicker in the case of ocular hypertension as compared to other types of glaucoma in which the value ranges from 513 to 570 microns. Lens thickness was significantly thicker in the ACG which ranges from 4.23 mm to 4.83 mm. The lens is relatively anteriorly positioned in the case of ACG calculated using the formula given by Lowe

(RLP=ACD+½LT/AL*10)

which ranges from 2.13 to 2.20. Lens axial-length factor was higher in the ACG in which the values ranged from 2.0 to 2.10. It is therefore recognised that the ocular dimensions are altered from a normal eye in the case of ACG than other types of glaucoma.

Accordingly, in some embodiments, the implant is dimensioned to be suitable for one or more of the following ocular dimensional parameters/eye parameters of a human eye in a diseased state (e.g., glaucoma, PACG): axial length, keratometry, anterior chamber depth, anterior chamber volume, lens thickness, pachymetry, relative lens position and len-axial length factor. In some embodiments, the implant is dimensioned to be suitable for a human eye with an axial length ranging from about 21 mm to about 23 mm, e.g., from about 21.62 mm to about 22.83 mm. In some embodiments, the implant is dimensioned to be suitable for a human eye with a corneal curvature ranging from about 43D to about 45D, e.g., from about 43.75D to about 44.87D, as measured with keratometry. In some embodiments, the implant is dimensioned to be suitable for a human eye with an anterior chamber depth ranging from about 1.8 mm to about 2.8 mm, e.g., from about 1.84 mm to about 2.69 mm. In some embodiments, the implant is dimensioned to be suitable for a human eye with an anterior chamber volume ranging from about 80 μl to about 120 μl. In some embodiments, the implant is dimensioned to be suitable for a human eye with a central corneal thickness ranging from about 500 μm to about 600 μm, e.g., from about 513 μm to about 570 μm. In some embodiments, the implant is dimensioned to be suitable for a human eye with a lens thickness ranging from about 4 mm to about 5 mm, e.g., from about 4.23 mm to about 4.83 mm. In some embodiments, the implant is dimensioned to be suitable for a human eye with a relative lens position ranging from about 2 to 2.3, e.g., from about 2.13 to about 2.20, as calculated using the formula given by Lowe

(RLP=ACD+½LT/AL*10).

In some embodiments, the implant is dimensioned to be suitable for a human eye with a lens axial-length factor ranging from about 1.9 to about 2.2, e.g., from about 2.0 to about 2.1.

As described in another study (Xinghuai Sun, Yi Dai, Yuhong Chen, Dao-Yi Yu, Stephen J. Cringle, Junyi Chen, Xiangmei Kong, Xiaolei Wang, Chunhui Jiang, Primary angle closure glaucoma: What we know and what we don't know, Progress in Retinal and Eye Research, Volume 57, 2017, Pages 26-45), a crowded anterior segment and narrow anterior chamber angle may be considered the basic anatomic features for PACG. The progression that the anterior chamber angle develops from narrow to become closed involves different factors. Iris thickness was shown to be likely to play a role in the development of angle closure and ultimately PACG. Smaller ACA (anterior chamber area) and ACV (anterior chamber volume) were independently associated with the presence of narrow angles, even after controlling for other known ocular biometric parameters, such as anterior chamber depth (ACD) and AL (axial length). Anterior chamber width (ACW), which was defined as the horizontal scleral spur-to-spur distance, could be a novel risk indicator for angle closure. ACV was calculated to be 25% lower in PACG patients than normal people. Accordingly, in some embodiments, the implant is dimensioned to be suitable for one or more of the following ocular dimensional parameters of PACG patients: iris thickness, ACA, ACV, ACD, AL and ACW.

As described in yet another study (Nongpiur M E, Sakata L M, Friedman D S, He M, Chan Y H, Lavanya R, Wong T Y, Aung T. Novel association of smaller anterior chamber width with angle closure in Singaporeans. Ophthalmology. 2010 October; 117 (10): 1967-73), variations in anterior chamber width (ACW) and its association with the presence of narrow angles were investigated. It was found that ACW was associated with narrow angle, independently of age, gender and AL, in two different studies of Singapore populations, suggesting that a smaller ACW may be a risk indicator for angle closure. The average anterior chamber width measured in the study was about 11.80 mm for normal patients versus 11.57 mm for patients with narrow angle.

Accordingly, in some embodiments, the implant is dimensioned to be suitable for PACG patients having an anterior chamber width ranging from about 11 mm to about 12 mm, e.g., an average anterior chamber width of about 11.57 mm.

In various embodiments, the implant may have dimensions that are configured for an Asian population, e.g., anatomical dimensions of an eye for an Asian population.

For example, in one study (Qin B, Tang M, Li Y, Zhang X, Chu R, Huang D. Anterior segment dimensions in Asian and Caucasian eyes measured by optical coherence tomography. Ophthalmic Surg Lasers Imaging. 2012; 43 (2): 135-142), a comparison was made between Asian and Caucasian anterior segment dimensions measured using optical coherence tomography (OCT). It was found that the anterior segments of the eye are smaller in Asian Americans compared to Caucasian Americans. Older subjects tend to have smaller anterior segment dimensions. These differences should be considered in the development of instruments and implants for the eye. Table 1 below is extracted from the study and provides a univariate analyses of eye dimensions and corneal power against race and gender.

TABLE 1
Univariate analyses of eye dimensions and corneal power against race and gender

| | Mean ± SD | | | Mean ± SD | | |
|---|---|---|---|---|---|---|
| Eye Parameters | Asian | Caucasian | P | Male | Female | P |
| Corneal diameter (mm) | 12.73 ± 0.43 | 13.23 ± 0.44 | <.01 | 13.09 ± 0.45 | 12.60 ± 0.82 | <.01 |
| AC width (mm) | 11.58 ± 0.40 | 12.04 ± 0.39 | <.01 | 11.90 ± 0.42 | 11.48 ± 0.73 | <.01 |
| Corneal vault (mm) | 2.89 ± 0.23 | 3.11 ± 0.24 | <.01 | 3.05 ± 0.18 | 2.83 ± 0.33 | <.01 |
| AC depth (mm) | 3.68 ± 0.35 | 3.87 ± 0.35 | .11 | 3.93 ± 0.36 | 3.57 ± 0.35 | <.01 |
| Corneal vault/diameter | 0.23 ± 0.013 | 0.24 ± 0.014 | .071 | 0.23 ± 0.010 | 0.22 ± 0.015 | <.01 |
| Axial length (mm) | 24.23 ± 1.18 | 24.02 ± 1.14 | .47 | 24.58 ± 1.28 | 23.61 ± 0.73 | <.01 |
| Limbal ellipticity (mm) | 0.13 ± 0.04 | 0.11 ± 0.03 | .072 | 0.12 ± 0.03 | 0.12 ± 0.04 | .28 |
| Corneal power (D) | 43.86 ± 1.56 | 44.20 ± 1.65 | .37 | 44.06 ± 1.49 | 44.14 ± 1.21 | .80 |

SD = standard deviation; AC = anterior chamber; D = diopters.

Accordingly, in some embodiments, the implant is dimensioned to be suitable for one or more of the following eye parameters of a human eye of a human subject, wherein the subject is an Asian or a Caucasian subject, or wherein the subject is a male or female subject: corneal diameter, AC width, corneal vault, AC depth, corneal vault/diameter, axial length, limbal ellipticity and corneal power. In some embodiments, the implant is dimensioned to be suitable for one or more eye parameters selected from Table 1, each eye parameter having a range of values with start and end points being defined by 1 standard deviation, 1.5 standard deviation, or 2 standard deviations from the mean value stated in Table 1.

The proximal segment of the elongate member may have a length ranging from about 0.5 mm to about 4 mm. The length of the proximal segment may be in a range with start and end points selected from the following group of numbers: 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, and 4 mm. In one embodiment, the length of the proximal segment is from about 0.5 mm to about 1 mm, such that the inlet port at the proximal end of the elongate member is not occluded by surrounding tissue. In another embodiment, the length of the proximal segment is from about 2 mm to about 4 mm, such that the proximal segment is sufficiently long to be seen/observable when the iris is dilated.

The distal segment of the elongate member may have a length ranging from about 1 mm to about 3 mm. The length of the distal segment may be in a range with start and end points selected from the following group of numbers: 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3 mm.

In various embodiments, the proximal segment and/or proximal tip of the implant comprises a specifically defined shape for augmenting the performance of the implant.

In various embodiments, the elongate member may be angled at its proximal tip such that the angled surface is configured to face away from a posterior surface/aspect of an iris, when the implant is deployed in the eye. For example, the elongate member may be bevelled at its proximal tip such that the bevelled surface is configured to face away from a posterior surface/aspect of an iris, when the implant is deployed in the eye. In other words, the proximal tip of the implant may comprise a bevel that is arranged to face away from the posterior aspect of the iris, when deployed in the eye. The bevelled proximal tip of the implant may advantageously minimise/reduce the likelihood of or prevent occlusion by the posterior surface of the iris. For example, conduction of fluid by the implant may still be possible when the iris presses on the proximal segment of the implant, so long as the bevelled proximal tip is faced away from the posterior surface of the iris. Advantageously, the bevel may also increase the cross-sectional luminal area at the proximal tip (c.f. with a non-bevelled circular cross-sectional lumen) through which fluid may pass, thereby facilitating drainage of fluid. The proximal tip of the implant may comprise a bevel angle of at least about 20°, at least about 25°, at least about 30°, at least about 35°, at least about 40°, or at least about 45°. The bevel angle is defined by the intersection between a longitudinal axis of the elongate member and the bevelled surface of the proximal tip of the elongate member. The bevel angle may also be in a range with start and end points selected from the following group of numbers: 20, 21, 22, 23, 24, 25, 26, 27, 28, 29, 30, 31, 32, 33, 34, 35, 36, 37, 38, 39, 40, 41, 42, 43, 44, and 45°.

The proximal tip of the implant may comprise edges that are blunted to avoid trauma to the iris. The blunted edges at the proximal tip of the implant may have a blunt radius at least 0.05 mm, at least 0.06 mm, at least 0.07 mm, at least 0.08 mm, at least 0.09 mm, or at least 0.10 mm.

In various embodiments, the distal segment and/or distal tip of the implant comprises a specifically defined shape for augmenting the performance of the implant.

In various embodiments, the distal tip of the implant may be tapered to facilitate entry of the implant at a site of implantation. In various embodiments, the distal tip of the implant may be blunted, e.g., having a substantially flattened/flat tip, to ensure that the distal tip does not cause additional trauma to surrounding tissues, e.g., cutting through the choroid tissue. The distal tip of the implant may have a blunt radius of no more than 0.1 mm, no more than 0.09 mm, no more than 0.08 mm, no more than 0.07 mm, no more than 0.06 mm, or no more than 0.05 mm.

In various embodiments, the distal segment of the elongate member may be substantially straight. In various embodiments, the distal segment of the elongate member may be curved with a radius of curvature that substantially matches, mimics or approximates a radius of curvature of an inner sclera of the eye. In various embodiments, the distal segment of the elongate member may be curved with a radius of curvature that is substantially similar or identical to a radius of curvature of an inner sclera of the eye. In one example, the distal segment of the implant has a curvature substantially identical to that of the sclera, and the distal tip is blunted, so that it does not pierce through the choroid tissue. In various embodiments, the distal segment of the implant may be curved with a radius of curvature that is larger than a radius of curvature of an inner sclera of the eye. In other words, the distal segment of the implant may have a radius of curvature that is larger than its expected path within the suprachoroidal space (in a longitudinal or circumferential direction). This allows the distal segment of the implant to slide within the suprachoroidal space along an inner surface of the sclera along its expected path and prevents re-entry of the distal tip of the elongate member through a choroid layer into a vitreous cavity of the eye.

In various embodiments, the implant comprises a lumen (i.e., inner lumen) disposed within and extending longitudinally along substantially an entire length of the elongate member. In various embodiments, the lumen functions to convey fluid from the inlet port at the proximal tip to the outlet port at the distal tip of the elongate member. The inner lumen may have a diameter of from about 0.1 to about 0.8 mm. The diameter of the lumen may be in a range with start and end points selected from the following group of numbers: 0.1, 0.15, 0.2, 0.25, 0.3, 0.35, 0.4, 0.45, 0.5, 0.55, 0.6, 0.65, 0.7, 0.75, and 0.8 mm. The implant may have a wall thickness that is dimensioned to maintain an entry profile that is sufficiently small, such that the implant can still pass over an access needle, pass through the ciliary tissue, pass through the opening formed in the choroid tissue by the access needle, and into the suprachoroidal space. The wall thickness of the lumen may be in a range of from about 0.05 to about 0.15 mm or with start and end points selected from the following group of numbers: 0.05, 0.06, 0.07, 0.08, 0.09, 0.1, 0.11, 0.12, 0.13, 0.14, and 0.15 mm. In one embodiment, the implant is capable of achieving a drainage flow rate of from about 2 μL/min to about 20 μL/min, which falls within the same order of magnitude as the physiological flow rate of aqueous humour in the eye. For example, the implant may comprise an inner lumen having a diameter of from about 0.1 mm to about 0.2 mm; an elongate member with a total length of from about 6 mm to about 8 mm and having a bent shape, and achieves a flow rate that of from about 2 μL/min to about 20 μL/min. The flow rate may be in a range with start and end points selected from the following group of numbers: 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, and 20 L/min. The range of flow rate of fluid through the implant may be achieved by adjusting the dimensions of the implant e.g., diameter of the inner lumen, length of the implant, angle of the bent shape etc.

In various embodiments, the implant comprises a cross-sectional profile that is arranged to maximize a surface area of the suprachoroidal space that is dissected. The implant may comprise one or more structural elements extending from an exterior surface of the elongate member, said structural elements being configured to maximize a surface area of the implant that is in contact with tissue in the suprachoroidal space. The structural elements may include a plurality of protrusions extending from the exterior surface of the elongate member. Each protrusion may comprise a lumen that is in fluid communication with the lumen of the elongate member, such that the lumens of the plurality of protrusions form an interconnected network of lumens for enhanced conduction of fluid. The structural elements may include plate-like or flange elements extending from the exterior surface of the elongate member, such that the implant may have a shape that is like a plate. The structural elements may include prong-like elements extending from the exterior surface of the elongate member, such that the implant may have a shape that is like a prong. The elongate member may be arranged to be in a curved configuration such that the implant has an S-shape.

The implant may have a width in the circumferential direction of more than about 0.1 mm, more than about 0.2 mm, more than about 0.3 mm, more than about 0.4 mm, more than about 0.5 mm, more than about 0.6 mm, more than about 0.7 mm, more than about 0.8 mm, more than about 0.9 mm, or more than about 1 mm. In one embodiment, the width of the implant in the circumferential direction is more than 0.5 mm.

The implant may be expandable to achieve a larger cross-sectional profile after deployment. In one embodiment, the implant may be made out of a wire structure/frame (e.g., coil, mesh, slotted tube) that is arranged to expand radially in a deployed state. During insertion, the implant may be maintained in an undeployed state, thereby allowing the implant to coapt axially around an access needle with a relatively smaller outer diameter. A smaller difference in diameter between the access needle and implant facilitates greater ease of implant extrusion, as there is less tissue that the tip of the implant has to push apart. Once the implant is in the right position, it may be expanded radially to its optimal dimensions in its deployed state.

In another embodiment, the radial expansion may be achieved by having a balloon over the access needle that is inflatable to deform the implant. The radial expansion may also be achieved by making the implant from a shape memory material such as Nitinol and locking it in its undeployed state (e.g., by having a sheath over it). The implant may be released after deployment.

In various embodiments, the implant is configured to be immobilised in a substantially fixed position, such that the implant does not substantially migrate after implantation.

The implant (e.g., the proximal segment or part thereof) may comprise one or more anchoring features that allow or promote retention of the implant within a ciliary tissue/muscle or a suprachoroidal space of the eye. The anchoring features may include but are not limited to elevated ridges, protruding legs, tabs or combinations thereof. It will be appreciated that in various embodiments, the structural elements that are configured to maximize a surface area of the implant that is in contact with tissue in the suprachoroidal space may also act as anchoring features.

The implant may comprise one or more topographical patterns formed on an exterior surface of the implant, thereby forming a patterned exterior surface. The one or more topographical patterns may be formed on the entire exterior surface of the implant or a portion thereof. The patterned surface may assist to provide increased grip, such that the implant is immobilised in a substantially fixed position and does not substantially or easily migrate after implantation.

The implant (e.g., distal segment or part thereof) may comprise one or more depressions or holes formed on an exterior surface of the implant to encourage tissue ingrowth such that the implant is immobilised in a substantially fixed position and does not substantially or easily migrate after implantation.

The implant (e.g., distal segment or part thereof) may comprise one or more textured portions on an exterior surface of the implant, thereby forming a rough exterior surface. The textured portions on the exterior surface may assist to encourage tissue ingrowth, such that the implant is immobilised in a substantially fixed position and does not substantially or easily migrate after implantation.

The implant may be configured to adopt a specific conformation after implantation such that the implant is immobilised in a substantially fixed position. The implant may also have eccentric curvature, meaning that the implant is curved in more than one plane (e.g., both in the sagittal and transverse planes).

In various embodiments, the implant is made of one or more biocompatible materials. Biocompatible materials may include but are not limited to: PTFE, ePTFE, SIBS, silicone, polyimide, Nitinol, stainless steel, titanium, polyurethane, siloxanes, etc or combinations thereof. The implant may comprise one or more types of coatings on its inner and outer surfaces. The coatings may advantageously modify the surface properties of the implant and confer useful properties such as anti-microbial, anti-fouling, anti-fibrotic, anti-inflammatory, biocompatibility, immune modulating properties etc. For example, the coatings may have anti-fouling properties such that the inner lumen, inlet port and outlet port do not become occluded over time. For example, the coatings may also possess anti-fibrotic properties to inhibit fibrosis.

The implant (e.g., proximal segment or part thereof) may have an external surface (e.g., external surface for contacting the surrounding tissue when implanted) that is substantially smooth such that it does not abrade the posterior surface of the iris. The surface of the implant may be substantially smooth with an average surface roughness of no more than about 0.1 microns, no more than about 0.2 microns, no more than about 0.3 microns, no more than about 0.4 microns, no more than about 0.5 microns, no more than about 0.6 microns, no more than about 0.7 microns, no more than about 0.8 microns, no more than about 0.9 microns, or no more than about 1 micron. In one embodiment, the surface of the implant may be substantially smooth with an average surface roughness of no more than about 0.5 microns.

The implant may have an internal surface (e.g., within the inner lumen) that is substantially smooth such that it does not become occluded (e.g., by biological matter such as particles suspended in aqueous humour passing through the inner lumen). The surface of the implant may be substantially smooth with an average surface roughness of no more than about 0.1 microns, no more than about 0.2 microns, no more than about 0.3 microns, no more than about 0.4 microns, no more than about 0.5 microns, no more than about 0.6 microns, no more than about 0.7 microns, no more than about 0.8 microns, no more than about 0.9 microns, or no more than about 1 micron. In one embodiment, the surface of the implant may be substantially smooth with an average surface roughness of no more than about 0.5 microns.

In various embodiments, the implant comprises one or more indicators/markers to facilitate positioning of the implant in the eye. The implant may comprise visual markings to facilitate the positioning of the implant. The visual markings may assist a user to determine whether the implant is inserted to a correct depth. In one embodiment, the proximal tip of the implant is coloured, such that it is easily visible/observable when the pupil is dilated.

In various embodiments, the implant is described in the form of a hollow elongate member. However, it will be appreciated that the form of the implant is not limited as such. In alternative embodiments, the implant may not have a hollow tube structure. In one example, the implant is not closed on all sides, but has a cross-sectional profile of a U-shaped channel. In another example, the implant possesses a wire scaffold structure, with the struts forming a channel for fluid to flow through.

In yet another example, the implant has more than one lumen, which may or may not be interconnected. In yet another example, the implant is porous and comprises multiple interconnected chambers. In yet another example, the implant has multiple inlets or outlets, taking the form of a U-shaped or Y-shaped structure.

### Deployer Tool

In various embodiments, there is provided a deployer/deployment tool for inserting an implant into an eye to facilitate conduction of fluid from a posterior chamber to a suprachoroidal space of an eye, the deployer tool comprising, a guide tube for accessing the posterior chamber of the eye through an insertion site, said guide tube comprising a distal tip that is configured to abut a ciliary sulcus or a portion of a ciliary body of the eye; and an access needle disposed within the guide tube and configured to create a delivery/guiding path from the sulcus or a portion of the ciliary body to the suprachoroidal space of the eye; wherein the deployer tool allows for movement of the implant e.g., through the guide tube in relation to the access needle along the delivery path such that a distal segment of the implant is disposed in the suprachoroidal space of the eye, and a proximal segment of the implant is disposed in the posterior chamber of the eye.

The deployer tool may be configured for deploying the implant between the posterior chamber of the eye and the suprachoroidal space of the eye. The implant may be a drainage shunt implant for draining aqueous humour, in the form of an elongate hollow member for maintaining a conduit between the posterior chamber and the suprachoroidal space of the eye, with a proximal end at the posterior chamber and a distal end in the suprachoroidal space.

In various embodiments, the deployer tool may further comprise an implant actuator configured to detachably couple to or engage the implant and configured to move the implant distally in relation to the access needle along the delivery path.

In various embodiments, the deployer tool further comprises a housing/casing. The housing may comprise an external surface for allowing an operator e.g., surgeon to hold/grip the deployer tool, and a chamber defined within the housing for holding various components of the deployer tool, e.g., guide tube, access needle and implant actuator. The housing may further comprise one or more outlets/openings for allowing various components of the deployer tool, e.g., guide tube, access needle and implant actuator, to extend outside of the housing. The various components of the deployer tool may be arranged to move independently of one another and/or to move in a coordinated manner within the housing. For example, the guide tube, access needle and implant actuator may be arranged concentrically along a common longitudinal axis within the housing such that one component can be separately controlled and moved independently of other components. The concentric arrangement along the common longitudinal axis within the housing may also facilitate coordinated e.g., simultaneous control and movement of two or more components along the longitudinal axis. Such an arrangement may advantageously allow independent and/or coordinated movement of the access needle and implant within the confined space of the deployer tool and operating space.

The deployer tool may be a hand-held tool. The housing may have a specific shape and/or form for ergonomic purposes that allows facile manipulation by an operator e.g., a surgeon. The deployer tool may comprise one or more controls e.g., electronic controls, physical controls (e.g., button, knob, slider, switch, lever, joystick etc.) disposed on an external surface of the housing. Each of the one or more controls may be associated with actuating a component of the deployer tool e.g., guide tube, access needle, and implant actuator. Actuation of a component may comprise controlling the movement, position and/or orientation of the component. Control of actuation may be physical, electronic, manual and/or automatic. For example, the deployer tool may have two sliding buttons disposed on the external surface, one of which is connected to an implant actuator holder and one of which is connected to an access needle holder. The housing of the deployer may be ergonomically configured to enhance user experience (e.g., ease of fine control, manipulation and handling) and improve comfort during use of the deployer tool. The housing of the deployer may be symmetric, hence allowing for both right and left-handed users. The one or more controls may be disposed on the external surface of the housing in a manner that is easy-to-reach and/or easy to push smoothly such that there is minimal or no need for the operator to reposition the holding/gripping position of the deployer tool.

Alternatively, the deployer tool may be coupled to or integrated into a system for performing robotic or robot-assisted surgery. For example, the deployer tool may be integrated into a robotic arm. Robotic surgery may be used to perform complex procedures with more precision, flexibility and control than is possible with conventional techniques. Robotic surgery is usually associated with minimally invasive surgery where the procedures are typically performed through tiny incisions.

### Guide Tube

In various embodiments, the guide tube functions as an external sheath for the implant, access needle and implant actuator. The guide tube may be in the form of an elongate member e.g., hollow elongate member having a distal end and a proximal end. The guide tube may comprise an opening (i.e., distal opening) disposed at the distal end of the elongate member for allowing the access needle and/or implant to extend and retract therefrom.

In various embodiments, the distal end/tip of the guide tube may be arranged to be abutted against a sulcus or a portion of a ciliary body. The abutment of the distal end/tip of the guide tube may provide feedback to an operator e.g., a surgeon that the distal end of the guide tube has reached the right place, either by visually observing tenting of the sclera or tactile detection. In one example, the abutment of the distal end/tip of the guide tube may cause a scleral region of the eye to tent outwards, thereby providing a visual signal/cue that the distal end of the guide tube has reached the right place. In another example, the guide tube and/or guide tube holder may be coupled to a tactile sensor e.g., a force sensor configured to generate a tactile feedback signal when the guide tube contacts a location in the eye e.g., at the sulcus or a portion of the ciliary body. The tactile feedback signal may be further converted into other forms of signals such as audio signals (e.g., beeping sound) and visual signal (e.g., light indicator).

In various embodiments, the guide tube is configured to have a Young's modulus that is sufficiently high such that it does not deflect when abutting the sulcus. For example, the guide tube may have a Young's modulus of at least about 7.5 GPa such that the guide tube does not substantially deflect when abutting the ciliary sulcus or a portion of the ciliary body of the eye. In various embodiments, the guide tube has a higher bending stiffness than the access needle and the implant. The access needle may be pre-formed in a bent configuration and configured to be straightened when retracted to be disposed within the guide tube. The guide tube may have a higher bending stiffness than the access needle such that the parts of the access needle that are within the guide tube has to substantially conform to its longitudinal shape (e.g., straight elongate member). Furthermore, in various embodiments, the guide tube does not bend or deflect when the access needle is retracted/pushed into it and the guide tube substantially maintains its shape.

In various embodiments, the distal end of the guide tube may be configured to have dimensions that substantially follow the shape of the ciliary sulcus, thereby allowing an approximate and reproducible fit when the device is placed in the correct position. The distal tip of the guide tube may be bent to substantially follow or mimic the shape of the ciliary sulcus. The distal tip of the guide tube may have a distal tip geometry with a reverse bevel angle of from about 10° to about 40°, and optionally a circumferential radius of from about 5 mm to about 7 mm. The guide tube/ciliary sulcus guide has an allowable width of less than 2 mm, so that it fits through a corneal incision. The reverse bevel angle may be in a range with start and end points selected from the following group of numbers: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40°. The circumferential radius may be in a range with start and end points selected from the following group of numbers: 5, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, and 7.0 mm.

In various embodiments, the distal tip of the guide tube may be blunted such that it does not substantially cause trauma to the posterior surface of the iris or the ciliary body. For example, the distal tip of the guide tube may be blunted with a corner radius of at least about 0.1 mm, at least about 0.2 mm, at least about 0.3 mm, at least about 0.4 mm, or at least about 0.5 mm. The distal tip of the guide tube may comprise inner edges that are chamfered or radiused, such that there are no burrs that may catch or attach to the implant as the implant is being released.

In various embodiments, the guide tube may have an inner surface that is substantially smooth such that it does not scratch or impede the sliding motion of the implant. For example, the inner surface of the guide tube may have a surface finish (or average surface roughness) of no more than about 0.1 microns, no more than about 0.2 microns, no more than about 0.3 microns, no more than about 0.4 microns, no more than about 0.5 microns, no more than about 0.6 microns, no more than about 0.7 microns, no more than about 0.8 microns, no more than about 0.9 microns, no more than about 1 micron, no more than about 2 microns, no more than about 3 microns, no more than about 4 microns, no more than about 5 microns, no more than about 6 microns, no more than about 7 microns, no more than about 8 microns, no more than about 9 microns, or no more than about 10 microns, such that it does not scratch or impede the sliding motion of the implant. This may be achieved by various types of treatment, such as chemical polishing, electropolishing etc.

### Guide Tube Holder

In various embodiments, the deployer tool further comprises a guide tube holder disposed thereon. The guide tube holder is configured to couple to or engage the proximal end of the guide tube. The guide tube holder may be arranged to be retained within the housing of the deployer tool. The guide tube holder may be arranged to be fixed or slidable within the housing of the deployer tool. The guide tube holder may be coupled to a control e.g., electronic control, physical control configured to actuate the guide tube, for allowing an operator to adjust the position of the guide tube holder and in turn, adjust the position of the guide tube relative to the housing. Control of actuation may be physical, electronic, manual and/or automatic.

### Access Needle

In various embodiments, the access needle functions to create the delivery path for the implant to follow from the deployer tool to a site of implantation, e.g., from a sulcus or a portion of a ciliary body to a suprachoroidal space of an eye. In various embodiments, the access needle comprises a distal end/tip and a proximal end/tip, a distal segment/portion and a proximal segment/portion. The access needle may be positioned/held within the implant. The access needle may be arranged to move linearly within the implant actuator. The access needle may be arranged to move linearly within the guide tube.

In various embodiments, the access needle is configured to access the suprachoroidal space of the eye by cutting/ablating tissue. In various embodiments, the distal tip of the access needle is configured to dissect/cut/ablate tissue e.g., tissue of the ciliary body so as to create the delivery path, wherein the delivery path comprises a first section extending from the sulcus or a portion of the ciliary body to an inner surface of a sclera and a second section extending from the inner surface of the sclera to the suprachoroidal space; and wherein the first section and second section form a bent angle along the delivery path. The distal end of the access needle may further be configured to dissect a suprachoroidal space of an eyeball. The distal end of the access needle may be configured to cut through the ciliary body but not the sclera, and then dissect posteriorly between the choroid layer and the sclera by following a curvature of the sclera. The distal end of the access needle may be configured to have a tip shape and sharpness that is enough to cut through the ciliary tissue but not the sclera.

In various embodiments, the access needle may be configured to form a bend from the ciliary sulcus into the suprachoroidal space, creating a delivery/guiding path that the implant can follow. The inventors have recognised that to create the delivery path, the access needle has to be configured such that the appropriate amount of localised cutting force is exerted, and that the access needle has to be sufficiently flexible to deflect in a posterior direction at the sclera e.g., by following a curvature of the sclera. The angle at which the access needle is required to bend when deflecting in the posterior direction at the sclera may be more acute than when the access needle is required to bend when being inserted from the anterior chamber of the eye. The creation of the delivery path by the access needle may depend on a few parameters such as the tip shape of the access needle, the profile at the distal segment of the access needle, the path taken by the access needle due to the constraint by the guide tube, the stiffness of the access needle, and the surface roughness of the access needle.

In various embodiments, the access needle has an outer diameter that is smaller than an inner diameter of the implant. The choice of using a small access needle with a larger bore implant (rather than a large access needle with a smaller implant) may advantageously create a conduit through the ciliary muscle that is as small and atraumatic as possible to minimise risk of damage to the surrounding tissue e.g., tissue of the ciliary body. The access needle may have an outer diameter ranging from about 100 μm to about 150 μm. The outer diameter of the access needle may be in a range with start and end points selected from the following group of numbers: 100, 102, 104, 106, 108, 110, 112, 114, 116, 118, 120, 122, 124, 126, 128, 130, 132, 134, 136, 138, 140, 142, 144, 146, 148, and 150 μm.

In various embodiments, the distal tip of the access needle is shaped to facilitate cutting and dissecting of tissue in the eye. The distal tip of the access needle may be tapered or bevelled. The distal tip of the access needle may be tapered with a taper angle of from about 30° to about 60°. The taper angle may be in a range with start and end points selected from the following group of numbers: 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60°. The distal tip of the access needle may be bevelled with a bevel angle of from about 30° to about 45°. The bevel angle may be in a range with start and end points selected from the following group of numbers: 30, 31, 32, 33, 34, 35, 36, 37, 38, 39, 40, 41, 42, 43, 44, and 45°. As the distal tip of the access needle contacts the sclera, the bevelled or tapered angles may facilitate a reaction force that is directed towards a posterior direction of the eye. This may help to guide the access needle posteriorly.

In various embodiments, the access needle is configured to be bendable to facilitate the creation of a curved delivery path for the implant to follow from the deployer tool to the site of implantation. The flexible access needle may be pre-bent at an angle or profile that substantially mimics or conforms to the profile of surrounding anatomical structures along the delivery path from the posterior chamber to the suprachoroidal space. The access needle may be configured such that both the proximal and distal portions are substantially straight; one of the proximal or distal portions is substantially straight and one of the proximal or distal portions is curved; or both the proximal and distal portions are curved.

In one embodiment, both the proximal and distal portions are substantially straight. The access needle may be configured to adopt a bent configuration, such that the distal portion is bent at from about 30° to about 50° with respect to the proximal portion, towards the lower surface of the deployer (that is facing the posterior direction of the eye). The bend radius may be from about 0.2 mm to about 1 mm. The distal portion of the access needle may range in length from about 1.5 to about 2.5 mm. The bend angle of the distal portion with respect to the proximal portion may be in a range with start and end points selected from the following group of numbers: 30, 31, 32, 33, 34, 35, 36, 37, 38, 39, 40, 41, 42, 43, 44, 45, 46, 47, 48, 49, and 50°. The bend radius may be in a range with start and end points selected from the following group of numbers: 0.2, 0.3, 0.4, 0.5, 0.6, 0.7, 0.8, 0.9, and 1.0 mm. The length of distal portion of the access needle may be in a range with start and end points selected from the following group of numbers: 1.5, 1.6, 1.7, 1.8, 1.9, 2.0, 2.1, 2.2, 2.3, 2.4, and 2.5 mm. The distal portion of the access needle is arranged to be pushed into the suprachoroidal space and may be sufficiently long to serve as a guiding path for the implant to bend into the suprachoroidal space. At the same time, the distal portion may be not too long that it increases the risk of cutting through the choroid layer. As the implant moves over the access needle, the implant shields the tip of the access needle, further lowering the risk of the access needle causing unwanted trauma to the tissue.

In another embodiment, the distal tip of the access needle follows a curved path, with a radius of curvature of from about 4 mm to about 6 mm. The radius of curvature of the curved path may be in a range with start and end points selected from the following group of numbers: 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, and 6 mm. The tangent of the distal tip may be at an angle of from about 30° to about 50° relative to the horizontal of the needle. The angle relative to the horizontal of the needle may be in a range with start and end points selected from the following group of numbers: 30, 31, 32, 33, 34, 35, 36, 37, 38, 39, 40, 41, 42, 43, 44, 45, 46, 47, 48, 49, and 50°.

In various embodiments, the path taken by the access needle is controlled by the guide tube. The guide tube may be shaped and/or dimensioned to constrain the access needle to a more precise delivery path. The access needle e.g., flexible access needle may be constrained as it leaves the guide tube such that it forms a path that can cut through the ciliary body and choroid layer and deflect posteriorly at the sclera.

In one embodiment, the outer diameter of the access needle may be configured to be within a predefined range of tolerance relative to an inner diameter of the guide tube, such that the access needle is substantially straightened inside the guide tube. As the access needle is extruded from the distal opening of the guide tube, the guide tube acts to constrain the degree to which the access needle can bend. As a result, the distal tip of the access needle forms a curved path as it is extruded, such that it traverses the ciliary body and dissects the suprachoroidal space. The inner diameter of the guide tube may be from about 0.1 mm to about 1 mm larger than the outer diameter of the access needle. The range of tolerance may be in a range with start and end points selected from the following group of numbers:

0.1, 0.2, 0.3, 0.4, 0.5, 0.6, 0.7, 0.8, 0.9, and 1.0 mm.

In another embodiment, the distal tip of the guide tube is bent or possesses an inner lumen with a curved configuration, in order to guide the motion of the access needle along a curved path into the suprachoroidal space.

In various embodiments, the access needle is configured to be substantially flexible. In various embodiments, the access needle may be pre-formed in a bent configuration and configured to be straightened when disposed within the guide tube and to return to the bent configuration when extended from the distal tip of the guide tube for the creation of the delivery path. In various embodiments, the access needle has a bending stiffness that is lower than that of the guide tube, such that the access needle does not deform e.g., bend or deflect the guide tube when the access needle is extended or retracted from the guide tube. The access needle may be made of a flexible material with shape memory properties, such as Nitinol, Nitinol alloys, copper-aluminium alloys and the like. As such, it retains its curvature despite passing through the straight portions of the guide tube e.g., ciliary sulcus guide.

In various embodiments, the stiffness of the access needle may be such that for an extruded length of at least 0.53 mm (length of the ciliary body), it can bend with an angle of at least 20 degrees with respect to its axis. In various embodiments, the stiffness of the access needle may be such that it can bend across a turning angle located at the inner sclera along the delivery path between the posterior chamber and the suprachoroidal space. In various embodiments, the access needle has a stiffness that is no more than about 2.5 N/mm to facilitate bending of the access needle at the bent angle along the delivery path. If the access needle is too stiff, it may pierce through the sclera, rather than bending posteriorly into the suprachoroidal space. In one embodiment, an access needle is made of superelastic Nitinol having a diameter of from about 0.13 mm to about 0.17 mm, such that the desired degree of stiffness is attained. In various embodiments, to prevent buckling of the access needle, the free length of the access needle (i.e., it is not supported by any external clamp or tube) is kept to less than 8 mm.

The access needle may also achieve flexibility by having flexible joints in the needle. This may be achieved by having multiple rigid segments of the access needle, connected by a flexible internal core.

In various embodiments, the access needle or a portion thereof is configured to have a substantially smooth surface finish to facilitate movement of the access needle through tissue in the eye. In various embodiments, the tapered or bevelled tips (i.e., sharpened faces) of the access needle may have a surface finish that allows the tip to be sharp enough to cut through the ciliary body and smooth enough to slide along the inner surface of the choroid. For example, the surface finish may be one where an average surface roughness (Ra) is no more than 0.5 micron, no more than 1 micron, no more than 2 microns, no more than 3 microns, no more than 4 microns, no more than 5 microns, no more than 6 microns, no more than 7 microns, no more than 8 microns, no more than 9 microns, or no more than 10 microns. In one embodiment, the surface finish of the sharpened faces is no more than 4 microns. The tapered or bevelled tip shapes may also facilitate atraumatic access through the ciliary body, such that bleeding is minimised. The surface finish of the tapered or bevelled tip may also be no more than 0.5 microns (Ra), to minimise tissue trauma. This may be achieved by various surface treatments, e.g., electropolishing, black oxide surface treatment or the like. Black oxide surface treatment may also help to prevent surface corrosion of the access needle. In various embodiments, the access needle comprises a feedback mechanism for enabling an operator e.g., surgeon to confirm whether the access needle has indeed entered the correct space, e.g., the suprachoroidal space. The feedback mechanism may be configured to detect a loss of resistance when the tip of the access needle e.g., flexible access needle travels through the ciliary muscle into the suprachoroidal space. For example, the access needle may be a hollow needle which permits the injection of fluid through the access needle. This can serve as a means of confirming whether the access needle has indeed entered the potential space between the two layers. If the access needle is in the right space, a loss of fluid resistance will be felt. This may be detected either in a tactile manner by the surgeon, or by using a pressure sensor.

In various embodiments, the implant is arranged to be retained within the guide tube as the access needle is pushed forward to create the delivery path from the deployer tool to a site of implantation. In various embodiments, the deployer tool is configured to extrude the implant after the delivery path is created by the access needle, such that the distal segment of the implant is disposed in the suprachoroidal space, and the proximal segment of the implant remains in the posterior chamber of the eye. The deployer tool may be configured such that as the access needle is being retracted from the site of implantation, the implant is retained (i.e., does not substantially move or migrate from its deployed position) and does not follow the access needle as it is being retracted.

It will be appreciated that in various embodiments, the access needle acts as an introducer to assist with delivery and placement of the implant at the site of implantation. In other embodiments, the deployer tool may comprise a separate introducer configured to assist with delivery and placement of the implant at the site of implantation. In one embodiment, the introducer takes the form of a hollow tube having a lumen, a proximal end/tip, and a distal end/tip. The introducer may have an inner diameter (i.e., diameter of the lumen) that is larger than the outer diameter of the implant and the access needle, such that the implant and the access needle may be disposable within the lumen of the introducer. The implant may be configured to be slidable within the lumen of the introducer. The distal tip of the introducer may be tapered or bevelled to facilitate its easy entry. The distal tip of the introducer may be maintained in an undeployed state with a relatively smaller cross-sectional profile to facilitate its insertion. The distal tip of the introducer may be maintained in a deployed state with a relatively larger cross-sectional profile at an entry location (e.g., an entry hole through the choroid) to facilitate deployment of the implant and prevent/minimise crumpling/scrunching up of the implant at the entry location. For example, the introducer may comprise one or more collapsible protrusions arranged radially on an external surface of the introducer at the distal tip. The one or more collapsible protrusions may be configured to open or spread outwards (e.g., to open up like a flower) to create the larger cross-sectional profile.

### Access Needle Holder

In various embodiments, the deployer tool further comprises an access needle holder disposed thereon. The access needle holder functions as a needle driver configured to move the access needle e.g., flexible access needle out of the guide tube and through a ciliary muscle of the eye into the suprachoroidal space. The access needle holder may be configured to couple to or engage the proximal end of the access needle. The access needle holder may be arranged to be retained within the housing of the deployer tool. The access needle holder may be arranged to be slidable within the housing of the deployer tool. The access needle holder may be coupled to a control e.g., electronic control, physical control, e.g., button, knob, or slider configured to actuate the access needle, for allowing an operator e.g., surgeon to adjust the position of the access needle holder and in turn, adjust the position of the access needle relative to the housing. The control may be disposed on the external surface of the housing of the deployer tool. Control of actuation may be physical, electronic, manual and/or automatic.

### Implant Actuator

In various embodiments, the deployer tool further comprises an implant actuator. In various embodiments, the implant actuator is configured to deploy (e.g., by pushing/advancing) the implant e.g., drainage shunt implant so that the distal segment of the implant is at the suprachoroidal space of the eye. The implant actuator may be arranged to be substantially parallel to and to move substantially linearly within the guide tube. The implant actuator may comprise a distal end and a proximal end. The distal end/face of the implant actuator may be arranged to detachably couple to or engage the implant. In other words, the distal end/face of the implant actuator may be arranged to engage (i.e., contact) and to disengage (i.e., break contact) with a portion (e.g., proximal end) of the implant. The distal end/face of the implant actuator may be arranged to push the implant out from the deployer along the delivery/guiding path created by the access needle.

In various embodiments, the implant actuator is configured to be slidably coupled to the access needle such that the implant actuator is arranged to slide along the access needle, e.g., along the delivery path created by the access needle. The implant actuator may be in the form of a hollow tube. The implant actuator may have an inner diameter that is larger than the diameter of the access needle, and smaller than the outer diameter of the implant. The inner diameter of the implant actuator may be from about 0.18 mm to about 0.25 mm. The inner diameter of the implant actuator may be in a range with start and end points selected from the following group of numbers: 0.18, 0.19, 0.20, 0.21, 0.22, 0.23, 0.24, and 0.25.

In various embodiments, the distal face of the implant actuator is arranged to abut the proximal end/face of the implant. The distal tip of the implant actuator may have a cross-sectional profile of a full circle having a hoop strength higher than that of the implant and does not get forced open by the implant. The distal tip of the implant actuator may have a shape that is complementary to the tip shape on the proximal tip of the implant. For example, the distal tip of the implant actuator may be bevelled upwards, with a bevel angle of from about 40° to about 50°, such that it is complementary to a bevelled tip shape on the proximal tip of the implant. This may advantageously facilitate control of the rotational orientation of the implant. Other suitable complementary fitting arrangements or engagements between the implant actuator and the implant may also be used.

In various embodiments, the implant actuator is configured to maintain the implant at the site of implantation so as to facilitate retraction of the access needle and/or guide tube. Once implanted, there should be minimal or no contact (i.e., direct physical contact) between the implant and the deployer on its inner and outer surfaces, so that the implant does not get dragged out with the deployer during retraction. The implant actuator may be configured to maintain the implant at the site of implantation (e.g., by maintaining the implant actuator at a fixed position relative to the housing) throughout the process of retraction.

In various embodiments, the access needle may be retracted relative to the implant actuator in a proximal direction, such that the distal tip of the access needle is of a minimum separation distance away from the proximal end of the implant actuator. The access needle may be retracted relative to the implant actuator in a proximal direction, such that the distal tip of the access needle is more proximal (i.e., at a more proximal position) in relation to the distal tip of the implant actuator. The guide tube may also be retracted relative to the implant actuator in the proximal direction, such that the distal tip of the guide tube is of a minimum separation distance away from the proximal end of the implant actuator. The guide tube may be retracted relative to the implant actuator in a proximal direction, such that the distal tip of the guide tube is more proximal (i.e., at a more proximal position) in relation to the distal tip of the implant actuator.

In various embodiments, the minimum separation distance away from the proximal end of the implant actuator may be at least about 0.2 mm, at least about 0.3 mm, at least about 0.4 mm, or at least about 0.5 mm. The access needle and/or guide tube may also be retracted fully, into the implant actuator such that there is no contact with the implant. The guide tube and access needle may be withdrawn sequentially one after another, or simultaneously for ease of use. The deployer tool may comprise one or more spring systems coupled to the access needle and guide tube to facilitate retraction into the housing.

In various embodiments, the implant actuator is arranged to decouple (i.e., break contact) from the implant after the implant is pushed into position. In various embodiments, the implant actuator or a portion thereof is configured to have a substantially smooth surface finish to facilitate disengagement/decoupling of the implant from the implant actuator after deployment, for e.g., such that the implant does not get undesirably/unintendedly caught onto the implant actuator. The surface finish of the distal face of the implant actuator may have an average surface roughness (Ra) of no more than 0.1 microns, no more than 0.2 microns, no more than 0.3 microns, no more than 0.4 microns, no more than 0.5 microns, no more than 0.6 microns, no more than 0.7 microns, no more than 0.8 microns, no more than 0.9 microns, no more than 1 micron, no more than 2 microns, no more than 3 microns, no more than 4 microns, no more than 5 microns, no more than 6 microns, no more than 7 microns, no more than 8 microns, no more than 9 microns, or no more than 10 microns.

### Implant Actuator Holder

In various embodiments, the deployer tool further comprises an implant actuator holder disposed thereon. The implant actuator holder may be configured to couple to or engage to the proximal end of the implant actuator. The implant actuator holder may be arranged to be retained within the housing of the deployer tool. The implant actuator holder may be arranged to be slidable within the housing of the deployer tool. The implant actuator holder may be attached to a control e.g., electronic control, physical control, e.g., button, knob, or slider configured to actuate the implant actuator, for allowing an operator e.g., surgeon to adjust the position of the implant actuator holder and in turn, adjust the position of the implant actuator relative to the housing. The control may be disposed on the external surface of the housing of the deployer tool. Control of actuation may be physical, electronic, manual and/or automatic.

### Tactile Feedback

In various embodiments, the deployer tool is configured to provide tactile feedback. The inventors have recognised that one difficulty associated with the implantation of an implant from the ciliary sulcus is that there is no direct visual feedback. The area of implantation is hidden because it is posterior to the iris. As such, the deployer tool may comprise features that allow for tactile feedback to an operator e.g., surgeon upon each step of implantation, such that the need for visualization is eliminated. Furthermore, it will be appreciated that excessive force should not be applied in pushing the access needle.

In one embodiment, the chamber of the housing may comprise an inner channel for allowing the access needle holder and implant actuator holder to slide freely within the inner channel. The inner channel of the housing may be configured such that the frictional forces between the access needle holder/implant actuator and the inner channel of the housing is less than 0.2 N, which is negligible as compared to the forces encountered at the distal tips of the access needle/implant actuator. This facilitates tactile feedback to an operator e.g., surgeon when the access needle cuts through the ciliary body, slides into the suprachoroidal space, and when the implant is pushed into the suprachoroidal space.

The inner channel of the housing may be substantially cylindrical. The access needle holder/implant actuator holder may have external faces that are substantially cylindrical, thereby allowing the access needle holder/implant actuator holder to be slidably fitted within the inner channel. The fit between the sliding elements and the housing may be within a tolerance of less than 0.1 mm. The materials of the housing and access needle holder/implant actuator holder may have low friction properties, such as Nylon, PC (polycarbonate), PEEK (polyetheretherketone) or the like. The surfaces of the inner channel and the access needle holder/implant actuator holder may be polished with a surface finish of less than about 1 microns (Ra) to permit free sliding.

The access needle, implant actuator, and guide tube may be arranged substantially concentrically along a common longitudinal axis, so as to ensure they can slide freely with respect to one another. The implant actuator and guide tube may have sufficient bending stiffness such that they remain straight with respect to one another and in minimal contact, rather than exerting a radial force that contributes to friction. For example, the implant actuator and guide tube may have sufficient bending stiffness of at least about 2000 MPa.

In another embodiment, the access needle holder, implant actuator holder may be coupled to a driving mechanism that follows the nut and bolt assembly of a micrometer screw gauge, where rotational movement of the proximal end leads to a controlled forward translation. For example, one complete rotation may translate to a linear distance equivalent to the pitch of the screw, i.e., the distance between two successive threads of a screw.

In yet another embodiment, the access needle holder, implant actuator holder may be coupled to a driving mechanism in the form of a plunger mechanism. The plunger mechanism may have features to assist with control of force applied, such as frictional resistance, or a spring or gas spring mechanism.

The following examples may provide alternative embodiments for ensuring feedback or confirmation of successful deployment. In one example, a portion of the deployment mechanism may be shaped as a slender member with a buckling strength below that required to puncture the sclera. If the force to push the needle is excessive, the portion of the deployment mechanism will buckle, resulting in an inability to advance further. In another example, the access needle may be arranged to contact the access needle holder e.g., needle driver with a sliding contact. If the force to push the needle is excessive, the access needle will be dislodged from the access needle holder. If the force is too high and the proximal end of the access needle bends, it will slide off a distal face of the access needle holder. In yet another example, the actuation mechanism may be coupled to a force sensor. If the force exerted is too high, some form of alert is activated, such as a visual indication (LED light comes on). Alternatively, the actuation mechanism may be driven by a motor, which gets cut off when the force detected by the force sensor exceeds the stipulated limit. The above approaches may be feasible due to the differential toughness of the choroid layer relative to the sclera.

### System for Facilitating the Conduction of Fluid

In various embodiments, there is provided a system e.g., delivery system for facilitating the conduction of fluid from a posterior chamber to a suprachoroidal space of an eye, the system comprising, an implant comprising, an elongate member having a proximal segment and a distal segment, wherein the elongate member is configured to maintain a bent configuration that substantially complies with anatomical curvatures of the posterior chamber and the suprachoroidal space, when deployed in the eye; and a deployer tool comprising, a guide tube for accessing the posterior chamber of the eye through an insertion site, said guide tube comprising a distal tip that is configured to abut a ciliary sulcus or a portion of a ciliary body of the eye; and an access needle configured to be disposed within the guide tube and to create a delivery path from the sulcus or a portion of the ciliary body to the suprachoroidal space of the eye; wherein the deployer tool allows for movement of the implant e.g., through the guide tube in relation to the access needle in a distal direction along the delivery path such that a distal segment of the implant is disposed in the suprachoroidal space of the eye, and a proximal segment of the implant is disposed in the posterior chamber of the eye.

In various embodiments, the elongate member is configured to be straightened to facilitate loading of the implant on a distal end of the access needle. The access needle may be configured to be extended such that its distal end is disposed outside of a housing of the deployer tool to facilitate loading. The implant may be pre-formed in a bent configuration and configured to maintain a bent configuration when deployed in the eye. The implant may be configured to be temporarily straightened to facilitate loading on the access needle.

In various embodiments, the implant and access needle are configured to be in a retracted position and disposed within the guide tube prior to deployment. In various embodiments, the distal end of the access needle and a distal end of the implant are configured to be positioned within the guide tube when in the retracted position. The access needle may be pre-formed in a bent configuration and configured to maintain a bent configuration when deployed in the eye to maintain the delivery path. The access needle and implant may be configured to be temporarily straightened to facilitate retraction and disposition within the guide tube.

In various embodiments, the insertion site is located on a cornea of the eye. In various embodiments, an incision e.g., clear corneal incision (CCI) is made on the cornea e.g., using a separate tool, prior to insertion of the guide tube. In various embodiments, the distal tip of the guide tube is configured to be inserted through the corneal insertion site into an anterior chamber of the eye, and to be inserted across a pupil of the eye, and to be moved along a posterior surface of an iris to abut the ciliary sulcus or a portion of the ciliary body of the eye. In various embodiments, the deployer tool may comprise one or more tactile feedback mechanisms configured to confirm the abutment of the distal tip of the guide tube against the sulcus or a portion of the ciliary body.

In various embodiments, the deployer tool further comprises an access needle holder configured to engage a proximal end of the access needle and configured to extend the access needle distally from the distal tip of the guide tube to allow a distal tip of the access needle to dissect tissue and create the delivery path. In various embodiments, the delivery path comprises a first section extending from the sulcus or a portion of the ciliary body to an inner surface of a sclera and a second section extending from the inner surface of the sclera to the suprachoroidal space; wherein the first section and second section form a bent angle along the delivery path.

In various embodiments, the deployer tool further comprises an implant actuator configured to detachably engage the implant; and an implant actuator holder configured to couple/engage the implant actuator and configured to move the implant actuator such that the implant actuator engages the implant, and the implant is moved distally in relation to the access needle along the delivery path. The implant and implant actuator may be configured to slide over the access needle along the delivery path until the implant reaches its intended position, with its distal segment in the suprachoroidal space and its proximal segment in the posterior chamber.

In various embodiments, the access needle holder is further configured to retract the access needle proximally such that the distal tip of the access needle is more proximal (i.e., in a more proximal position) in relation to the distal tip of the implant actuator.

In various embodiments, the deployer tool may further comprise a guide tube holder configured to couple/engage the guide tube and configured to move the guide tube in relation to the housing. The guide tube holder may be configured to retract the guide tube proximally such that the distal tip of the guide tube is more proximal (i.e., in a more proximal position) in relation to the distal tip of the implant actuator. The guide tube holder may be configured to retract the guide tube concurrently or sequentially with the retraction of the access needle.

In various embodiments, the guide tube holder, access needle holder and implant actuator holder may be configured to actuate in a substantially linear direction using the one or more controls disposed on the housing of the deployer tool. The guide tube holder, access needle holder and implant actuator holder may be configured to be positioned in a fully retracted state where the guide tube, access needle holder and implant actuator holder are positioned at their most proximal positions within the housing. The guide tube holder, access needle holder and implant actuator holder may be configured to be positioned in a fully extended state where the guide tube, access needle holder and implant actuator holder are positioned at their most distal positions within the housing. The guide tube holder, access needle holder and implant actuator may further be arranged move between their most distal and most proximal positions within the housing.

In various embodiments, one or more components of the implant, deployer tool and system is/are biocompatible, bioinert, and sterilisable. In various embodiments, parts of the implant, deployer tool and system that come into contact (directly or indirectly) with the eye is/are biocompatible, bioinert, and sterilisable. In various embodiments, the whole implant and/or the whole deployer tool is/are fully is/are biocompatible, bioinert, and sterilisable. Sterilisation may be performed using suitable materials and techniques known in the art, e.g., ethylene oxide, gamma or electron beam irradiation, plasma, or autoclave sterilization. Accordingly, one or more components of the implant, deployer tool and system is/are made of materials that are able to withstand the desired sterilization methods without substantial or appreciable loss in their desired properties or functions.

Method of Inserting an Implant into an Eye

In various embodiments, there is provided a method of inserting an implant into an eye to facilitate conduction of fluid from a posterior chamber to a suprachoroidal space of an eye, the method comprising, inserting a guide tube through an insertion site such that a distal tip of the guide tube is disposed in the posterior chamber of the eye; abutting the distal tip of the guide tube on a sulcus or a portion of a ciliary body of the eye; advancing an access needle to create a delivery path from the sulcus or a portion of the ciliary body to the suprachoroidal space of the eye; and moving the implant in relation to the access needle along the delivery path, such that a distal segment of the implant is disposed in the suprachoroidal space of the eye and a proximal segment of the implant is disposed in the posterior chamber of the eye.

In various embodiments, the method is performed using a specially designed deployer tool. The deployer tool is substantially similar in structure and function to the deployer tool in various embodiments as disclosed herein. The implant is substantially similar in structure and function to the implant in various embodiments as disclosed herein. The deployer tool may comprise a housing with an external surface for allowing an operator to hold/grip the deployer tool, and a chamber defined within the housing for holding the guide tube, access needle, implant actuator and other components of the deployer tool. The deployer tool may further comprise a guide tube holder, an access needle holder and an implant actuator holder, each coupled to a proximal end of the guide tube, access needle and implant actuator, respectively. The guide tube holder, access needle holder and implant actuator holder may be further coupled to one or more controls e.g., electronic controls, physical controls (e.g., button, knob, slider, switch, lever, joystick etc.) for allowing the operator to actuate or to control movement and positions of the guide tube, access needle and implant actuator. The one or more controls may be disposed on the external surface of the housing in an easy-to-reach or ergonomic manner, such that there is minimal or no need for the operator to reposition or adjust the holding/gripping position of the deployer tool.

In various embodiments, the method further comprises, prior to insertion at the insertion site, loading the implant on the access needle. Loading of the implant may comprise actuating the access needle holder to extend the access needle outside of the housing of the deployer tool to facilitate loading. The implant may be configured to maintain a bent configuration when deployed/implanted in the eye. Loading of the implant may involve temporary straightening of the implant so as to conform to the shape or profile of the access needle.

In various embodiments, the method further comprises, after loading the implant, retracting the access needle and implant actuator such that a distal end of the access needle and a distal end of the implant are positioned within the guide tube.

In various embodiments, the method comprises inserting the guide tube through the insertion site such that the distal tip of the guide tube is disposed in the posterior chamber of the eye. The insertion site may be a location on the cornea of the eye. Inserting the guide tube through the insertion site may comprise making an incision in the cornea to allow the guide tube to pass through. Inserting the guide tube through the insertion site may further comprise inserting the guide tube through the cornea into an anterior chamber of the eye and moving the guide tube across a pupil of the eye to access the posterior chamber. Inserting the guide tube through the insertion site may comprise moving the entire housing of the deployer tool while being held and manipulated by the operator, such that the distal tip of the guide tube is inserted through the corneal insertion into the anterior chamber and crosses the pupil to reach the posterior chamber.

In various embodiments, the method comprises abutting the distal tip of the guide tube on the sulcus or a portion of the ciliary body of the eye. This may comprise navigating/moving the guide tube under an iris of the eye until the distal tip of the guide tube locates and abuts the sulcus or a portion of the ciliary body of the eye. The abutment of the distal tip of the guide tube against the sulcus or a portion of the ciliary body may be confirmed using one or more tactile feedback mechanisms provided by the deployer tool.

In various embodiments, the method comprises advancing the access needle to create the delivery path from the sulcus or a portion of the ciliary body to the suprachoroidal space of the eye. This may comprise extending the distal tip of the access needle from the distal tip of the guide tube and dissecting tissue at the ciliary sulcus or the portion of the ciliary body where the distal tip of the guide tube is abutted. The access needle holder may be advanced such that the access needle moves distally, cutting through the ciliary body. The step of advancing the access needle to create the delivery path may further comprise advancing the access needle to contact an inner aspect of a scleral wall of the eye, sliding the access needle along the inner aspect of the scleral wall of the eye, and dissecting the suprachoroidal space of the eye. In various embodiments, the access needle does not cut the scleral wall of the eye, i.e., the scleral wall is spared.

In various embodiments, the method comprises advancing an implant actuator to move the implant distally in relation to the access needle, such that the distal segment of the implant is disposed in the suprachoroidal space of the eye and the proximal segment of the implant is disposed in the posterior chamber of the eye. Advancing the implant actuator to move the implant may comprise advancing the implant actuator holder such that the implant actuator also advances and pushes the implant distally. Advancing the implant actuator to move the implant may further comprise sliding the implant over the access needle along the delivery path until the implant reaches its intended position, with its distal segment in the suprachoroidal space and its proximal segment in the posterior chamber.

In various embodiments, the method further comprises withdrawing the deployer tool from the site of insertion e.g., corneal incision, leaving the implant in place. This may comprise retracting the access needle holder such that the access needle is retracted until its distal tip is more proximal in relation to the distal tip of the implant actuator. The access needle may be retracted in a proximal direction along the delivery path until the distal tip of the access needle is decoupled from a proximal tip of the implant. Withdrawing the deployer tool from the site of insertion may further comprise retracting the guide tube holder such that the guide tube is retracted until its distal tip is more proximal in relation to the distal tip of the implant actuator. The retraction of the guide tube may be done concurrently with the retraction of the access needle.

In various embodiments, the method of inserting the implant may provide an ab-interno approach for the insertion of a shunt directed from the ciliary sulcus behind the posterior iris, through the ciliary muscle while avoiding the vascularized ciliary processes, and finally into the potential suprachoroidal space.

In various embodiments, the method of inserting the implant may advantageously provide an improved way to create and maintain an outflow drainage tract from the posterior chamber to the suprachoroidal space of the eye, for drainage of aqueous humour, hence augmenting the physiologic uveoscleral outflow pathway. Embodiments of the method as disclosed herein may be categorised as a form of minimally invasive glaucoma surgery (MIGS), with an aim of improving treatment efficacy while maintaining a safety profile not more than lens extraction/phacoemulsification alone.

In various embodiments, the method of inserting the implant may advantageously address the issues associated with existing MIGS devices and issues which are relevant to chronic angle closure glaucoma. In particular, the method as disclosed herein may avoid an approach superior to the iris via the narrow iridocorneal angle, instead opting for the larger retro iridial space that is created upon lens removal. Embodiments of the method as disclosed herein may augment the uveoscleral outflow pathway, which is less prone to possible dysfunction as compared to the TM/SC pathway. The method as disclosed herein may bypass any re-formed anterior PAS or iris occlusion, hence providing a route for outflow drainage despite any anterior irido-trabecular contact. Embodiments of the method as disclosed herein may entail less risk of chronic endothelial cell loss as compared to an implant retained in the anterior chamber, as the iris serves as a barrier between the implant and the corneal endothelium. Embodiments of the method as disclosed herein may lead to less scarring and fibrosis as compared with subconjunctival outflow and/or ab-externo approaches.

Furthermore, in various embodiments of the method disclosed herein, there is also potential for greater efficacy (i.e., IOP reduction or medication reduction) compared to TM/SC approaches, as the degree of pressure lowering is not limited by the episcleral venous pressure.

The inventors have recognised that the embodiments of the method as disclosed herein may include some unique considerations. Firstly, conventional visualization of the ciliary sulcus area is not possible due to the overlying iris, and entry from the ciliary sulcus would include careful and deliberate positioning in order to avoid damage to the adjacent ciliary processes and iris root which would lead to bleeding or hyphema. Secondly, to maintain an ab-interno approach with no scleral dissection, access to the suprachoroidal space from the ciliary sulcus may be achieved by selective cutting or ablation through the superficial or proximal layer of tissue (i.e., choroid and/or ciliary muscle/tissue), without cutting the deep/distal layer (i.e., inner aspect of the sclera). Thirdly, due to the relative positioning of the ciliary sulcus, ciliary body, and suprachoroidal space, the implant possesses a bend in its deployed configuration, and by extension the delivery mechanism may serve to enable this.

The inventors have also recognised that embodiments of the method as disclosed herein may not be achieved safely and reproducibly with currently available devices and surgical tools within a reasonable operative time. As such, various embodiments of the implant and deployer tool disclosed herein may advantageously address the challenges inherent to this approach, by providing a ciliary sulcus to suprachoroidal drainage shunt (implant) and surgical tools (deployer tool) that allows for its successful placement ab-interno.

Various embodiments of the implant, deployer tool, system and method disclosed herein may provide a MIGS approach for chronic angle closure glaucoma patients that address both issues of drainage and potential for rePAS (reformation of peripheral anterior synechiae). Various embodiments of the implant, deployer tool, system and method disclosed herein may facilitate access to the suprachoroidal space via the ciliary sulcus in an ab-interno fashion. Various embodiments of the implant, deployer tool, system and method disclosed herein may enable successful deployment of the implant by having one or more of the following characteristics: (i) providing a delivery path e.g., curved access path from the ciliary sulcus to the suprachoroidal space; (ii) selective cutting/ablation of the ciliary tissue, while sparing the sclera; (iii) providing a curved or bent implant in its deployed configuration; (iv) customising the shape, dimensions, and sharpness of the access needle that allows for cutting through choroid tissue but not the sclera; (v) customising the implant material and dimensions that give it sufficient flexibility to follow the curved path of the access needle into the suprachoroidal space; and (vi) customising the dimensions of the distal portions of the implant and access needle, that minimise the chances of either cutting through the choroid tissue into the vitreous body.

### BRIEF DESCRIPTION OF FIGURES

FIG. 1 is a schematic drawing of an eye with an implant e.g., drainage shunt implant inserted therein in an example embodiment.

FIG. 2A is a schematic cross-sectional view drawing of a deployer/deployment tool for inserting an implantable shunt for creating and maintaining an outflow drainage tract from a posterior chamber to a suprachoroidal chamber of an eye in an example embodiment.

FIG. 2B is a perspective view drawing of the deployer/deployment tool in the example embodiment.

FIG. 3 is a schematic cross-sectional side view drawing of a deployer tool in another example embodiment.

FIG. 4A is a schematic cross-sectional side view drawing of a guide tube of a deployer tool in an example embodiment.

FIG. 4B is a schematic cross-sectional side view drawing of the guide tube positioned at a sulcus of a ciliary body of an eye in the example embodiment.

FIG. 5 is a schematic perspective view drawing of a portion of an access needle in a first example embodiment.

FIG. 6 is a schematic perspective view drawing of a portion of an access needle in a second example embodiment.

FIG. 7 is a schematic view drawing of an access needle in a third example embodiment.

FIG. 8 is a schematic view drawing of an access needle in a fourth example embodiment.

FIG. 9A is a first schematic cross-sectional view of a portion of a deployer tool in an example embodiment.

FIG. 9B is a second schematic cross-sectional view of the portion of the deployer tool in the example embodiment.

FIG. 9C is a third schematic cross-sectional view of the portion of the deployer tool in the example embodiment.

FIG. 9D is a fourth schematic cross-sectional view of the portion of the deployer tool in the example embodiment.

FIG. 10 is a schematic cross-sectional view drawing of an implant e.g., drainage shunt implant in a first example embodiment.

FIG. 11 is a perspective view drawing of an implant e.g., drainage shunt implant in a second example embodiment.

FIG. 12 is a perspective view drawing of an implant e.g., drainage shunt implant in a third example embodiment.

FIG. 13 is a perspective view drawing of an implant e.g., drainage shunt implant in a fourth example embodiment.

FIG. 14 is a schematic side view drawing of an implant e.g., drainage shunt implant inserted in an eye in an example embodiment.

FIG. 15 is a schematic side view drawing of an implant e.g., drainage shunt implant inserted in an eye in another example embodiment.

FIG. 16 is a side view drawing of an implant e.g., drainage shunt implant in a fifth example embodiment.

FIG. 17 is a perspective view drawing of an implant e.g., drainage shunt implant in a sixth example embodiment.

FIG. 18 is a perspective view drawing of an implant e.g., drainage shunt implant in a seventh example embodiment.

FIG. 19 is a perspective view drawing of an implant e.g., drainage shunt implant in an eighth example embodiment.

### DETAILED DESCRIPTION OF FIGURES

Example embodiments of the disclosure will be better understood and readily apparent to one of ordinary skill in the art from the following discussions and if applicable, in conjunction with the figures. It should be appreciated that other modifications related to structural, material and optical changes may be made without deviating from the scope of the invention. Example embodiments are not necessarily mutually exclusive as some may be combined with one or more embodiments to form new exemplary embodiments. The example embodiments should not be construed as limiting the scope of the disclosure.

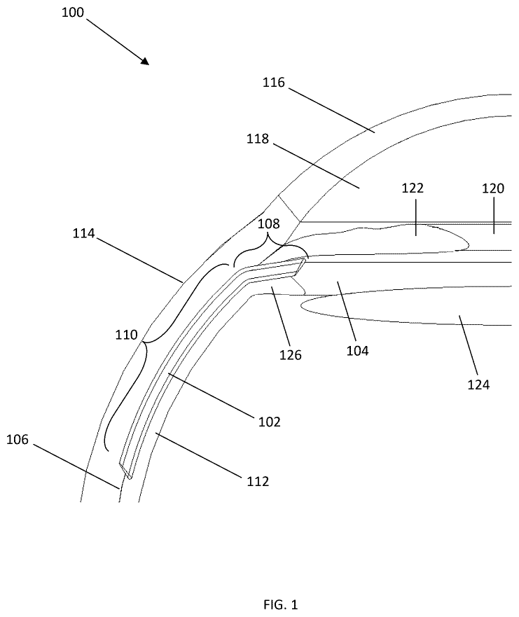

FIG. 1 is a schematic drawing of an eye 100 with an implant e.g., in the form of a drainage shunt implant 102 inserted therein in an example embodiment. The drainage shunt functions to maintain an outflow drainage tract from a posterior chamber 104 to a suprachoroidal space 106 of the eye. The drainage shunt implant 102 takes the form of an elongate hollow member having a proximal segment 108 disposed in the posterior chamber 104 of the eye 100 and a distal segment 110 disposed in the suprachoroidal space 106 of the eye 100. The suprachoroidal space is a space that is between a choroid 112 and a sclera 114 that traverses the circumference of the posterior segment of the eye 100. The drainage shunt implant 102 maintains a bent configuration that substantially complies with the anatomical curvature of the suprachoroidal space 106, when deployed in the eye.

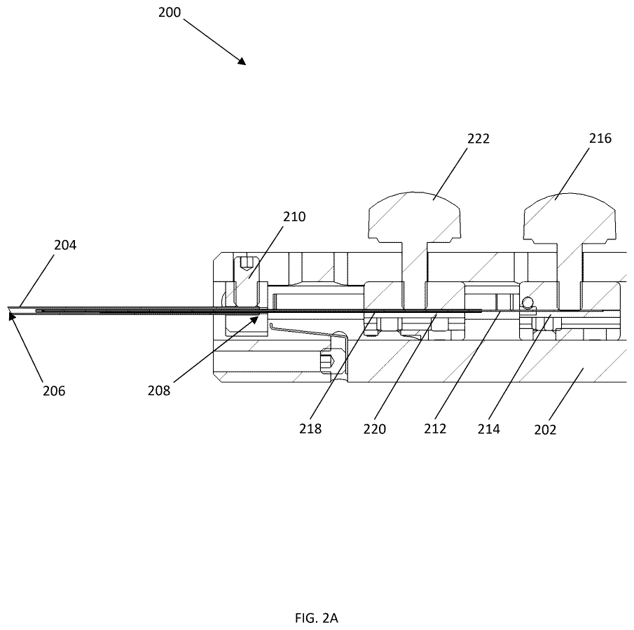

In the example embodiment, the drainage shunt implant 102 is inserted into position using an ab-interno approach, wherein the drainage shunt implant 102 is delivered along a delivery/guiding path created by a deployer tool. The drainage shunt implant 102 is advanced along the delivery path, moving through an insertion site on a cornea 116 to an anterior chamber 118, moving across a pupil 120 to enter the posterior chamber 104, moving between a posterior aspect of an iris 122 and an intraocular len 124 to a sulcus of a ciliary body 126, moving through tissue e.g., muscle of the ciliary body 126 towards an inner aspect of the sclera 114 while avoiding the vascularized ciliary processes, and moving in the suprachoroidal space 106 towards a posterior aspect of the eye 100 along a curvature of the sclera 114. FIG. 2A is a schematic cross-sectional view drawing of a deployer/deployment tool 200 for inserting an implantable shunt for creating and maintaining an outflow drainage tract from a posterior chamber to a suprachoroidal chamber of an eye in an example embodiment. FIG. 2B is a perspective view drawing of the deployer/deployment tool 200 in the example embodiment. The implantable shunt may have a form of an elongate hollow member, with a proximal end that is to be positioned at the posterior chamber and a distal end that is to be positioned in the suprachoroidal space of the eye.

As shown in FIG. 2A, the deployer tool 200 comprises a housing 202 in the form of an elongate member having an external surface for allowing an operator e.g., surgeon to hold the deployer tool 200, an internal chamber for housing various components e.g., sliding elements of the deployer tool 200, and an opening for allowing various components e.g., sliding elements to extend outside the housing 202.

The deployer tool 200 further comprises a guide tube 204 in the form of a hollow elongate member having a distal end 206 with an opening and a proximal end 208 coupled/attached to a guide tube holder 210. The guide tube holder 210 is retained within the housing 202 and can be either fixed to or sliding within the housing 202.

The deployer tool 200 further comprises an access needle 212 in the form of an elongate member having a distal end and a proximal end. The distal end is configured to cut through a ciliary body and optionally to dissect a suprachoroidal space of an eye. The proximal end of the access needle 212 is coupled/fixed to an access needle holder 214 which is retained within the housing 202 and is arranged to be slidable within the housing 202. The access needle holder 214 is attached to a first control e.g., first slidable knob 216 which can be actuated by the operator to control the position of the access needle holder 214 and the access needle 212.

The deployer tool 200 further comprises an implant actuator 218 having a distal end and a proximal end. The distal end is used to push the drainage shunt implant out from the deployer 200. The proximal end of the implant actuator 218 is coupled/fixed/secured to an implant actuator holder 220 which is retained within the housing 202 and is arranged to be slidable within the housing 202. The implant actuator holder 220 is attached to a second control e.g., second slidable knob 222 which can be actuated by the operator control the position of the implant actuator 218 and the implant actuator holder 220.

The guide tube 204 serves as an external sheath for the implantable shunt, access needle 212, and implant actuator 218. The implant actuator 218 is substantially parallel to and moves linearly within the guide tube 204. The access needle 212 is substantially parallel to and is arranged to move linearly within the implant actuator 218. The access needle 212 and implantable shunt are arranged to exit from the opening at the distal end 206 of the guide tube 204.

As shown in FIG. 2B, the housing 202 is configured such that it is ergonomic, and the buttons e.g., 216, 222 are easy to push smoothly without having to change the grip. This allows for the deployer tool 200 to be held steadily during a procedure for inserting the implantable shunt. The deployer tool 200 has two sliding controls 216, 222 arranged in line on the top surface of the housing 202, one of which is connected to the access needle holder 214 and one of which is connected to the implant actuator holder 220. The housing 202 is symmetric, hence allowing for both right- and left-handed users.

The deployer/deployment tool 200 may be used in an implantation procedure for creating and maintaining an outflow drainage tract from a posterior chamber to a suprachoroidal chamber of an eye using an ab-interno approach. The implantation procedure may comprise the following steps:
(1) The implant is loaded on the access needle.
(2) The access needle and implant actuator are at the most proximal position. The access needle and implant are fully within the guide tube holder (in a linear direction).
(3) The deployer is inserted through a corneal insertion into the anterior chamber by moving the entire housing. The guide tube crosses the pupil, moves under the iris, until the distal tip abuts the sulcus or a portion of the ciliary body.
(4) The access needle holder is advanced such that the access needle moves distally, cutting through the ciliary body.
(5) The access needle holder is further advanced such that the access needle contacts the inner aspect of the scleral wall and slides posteriorly along it, dissecting the suprachoroidal space.
(6) The implant actuator holder is advanced, such that the implant actuator also advances and pushes the implant distally.
(7) The implant slides over the access needle into position, with its distal segment in the suprachoroidal space and it proximal segment in the posterior chamber
(8) The access needle holder is retracted such that the access needle is retracted until its distal tip is proximal to the distal tip of the implant actuator.
(9) Optionally, the guide tube holder is retracted such that the guide tube is retracted until its distal tip is proximal to the distal tip of the implant actuator. This can be done concurrently with the previous step (8).
(10) The deployer is withdrawn through the corneal incision, leaving the implant in place.

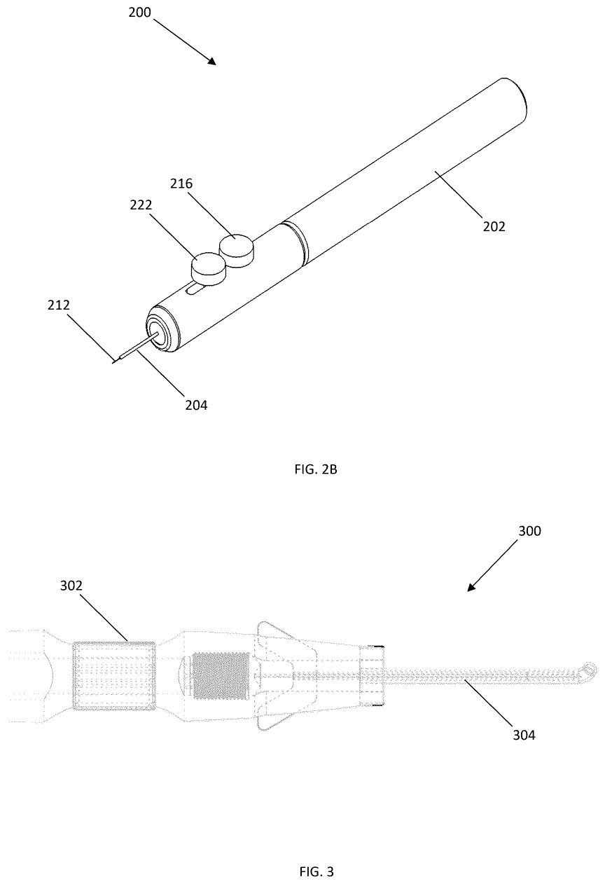

FIG. 3 is a schematic cross-sectional side view drawing of a deployer tool 300 in another example embodiment. The deployer tool 300 functions substantially similarly to the deployer tool 200. The deployer tool 300 comprises a nut and bolt assembly 302 coupled to an access needle 304. Unlike the sliding controls used in the deployer 200 of FIGS. 2A and 2B, the deployer tool 300 utilises an alternative needle driver mechanism that follows the nut and bolt assembly of a micrometer screw gauge, where rotational movement of the proximal end leads to a controlled forward translation. One complete rotation translates to a linear distance equivalent to the pitch of the screw, i.e., the distance between two successive threads of a screw.

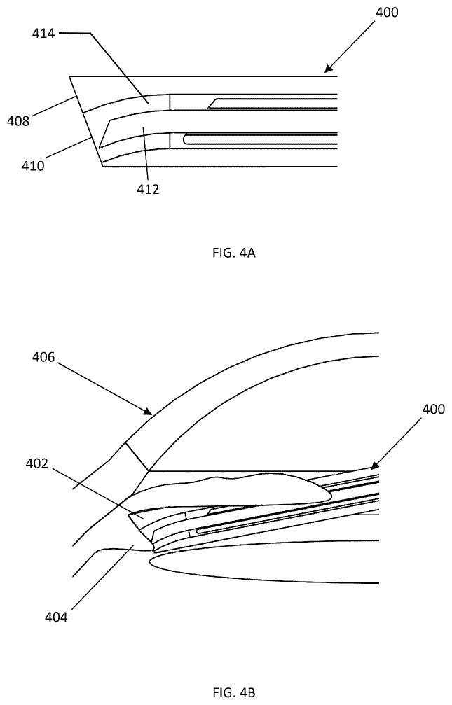

FIG. 4A is a schematic cross-sectional side view drawing of a guide tube 400 of a deployer tool in an example embodiment. FIG. 4B is a schematic cross-sectional side view drawing of the guide tube 400 positioned at a sulcus 402 of a ciliary body 404 of an eye 406 in the example embodiment.

The configuration of the guide tube 400 plays a role in the correct placement of the deployer tool at the sulcus 402 of the ciliary body 404. The guide tube 400 comprises a distal tip 408 that is configured to abut against the sulcus 402 or a portion of the ciliary body 404 and an opening 410 for allowing an access needle 412 to extend from or withdraw into the guide tube 400. This allows for feedback to an operator e.g., surgeon that the distal tip 408 of the guide tube 400 has reached the right place/position, either by visually observing the tenting of the sclera or tactile detection.

The dimensions of the distal end 408 of the guide tube 400 follow the shape of the ciliary sulcus 402, allowing an approximate and reproducible fit when the deployer tool/device is placed in the correct position. Specifically, this is embodied by a distal tip geometry with a reverse bevel angle of from about 10° to about 40°, and optionally a circumferential radius of from about 5 mm to about 7 mm. The allowable width of a ciliary sulcus guide is less than 2 mm, so that it fits through the corneal incision.

In the example embodiment, the distal tip 408 of the guide tube 400 is shown to be substantially straight with a curved inner lumen 414, in order to guide the motion of the access needle 412 along a curved path into the suprachoroidal space. In other embodiments, the distal tip 408 of the guide tube 400 may be bent and/or possess an inner lumen with a curved configuration.

The distal tip of the guide tube 400 may be blunted with a corner radius of more than 0.1 mm, such that it does not cause trauma to the posterior surface of the iris or the ciliary body. The guide tube 400 may have a Young's modulus of more than 7.5 GPa, such that it does not deflect when abutting the sulcus (not shown) of the eye 406.

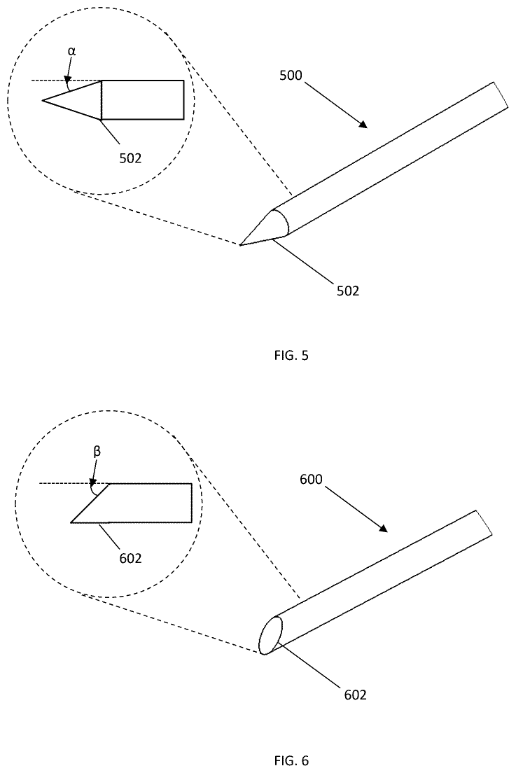

FIG. 5 is a schematic perspective view drawing of a portion of an access needle 500 in a first example embodiment. FIG. 6 is a schematic perspective view drawing of a portion of an access needle 600 in a second example embodiment. The configuration of the access needles plays a role in the dissection dynamics of tissue for insertion of an implant e.g., drainage shunt implant. The access needles 500, 600 are configured to cut through the ciliary body but not the sclera of an eye, and then dissect posteriorly between the choroid layer and sclera of the eye. In the end state, the access needles 500, 600 are arranged to form a bend from the ciliary sulcus into the suprachoroidal space, creating a delivery/guiding path that an implant e.g., drainage shunt implant can then follow.

The creation of the delivery path depends on a few parameters such as the tip shape of the access needle, the profile at the distal segment of the access needle, the path taken by the access needle due to the constraint by the guide tube, stiffness, and surface roughness of the access needle.

The access needles 500, 600 each has an outer diameter ranging from about 100 μm to about 150 μm. The outer diameter of the access needles 500, 600 are smaller than the inner diameter of the implant.

As shown in the figures, the first access needle 500 has a distal tip 502 with a taper angle α of 30 to 60 degrees (see side view drawing of the distal tip 502). The second access needle 600 has a bevelled tip 602 with a bevel angle β of 45 degrees (see side view drawing of the bevelled tip 602). As the distal tip of the access needle contacts the sclera, the bevelled or tapered angles can facilitate a reaction force that is directed towards the posterior of the eye. This helps to guide the access needle posteriorly.

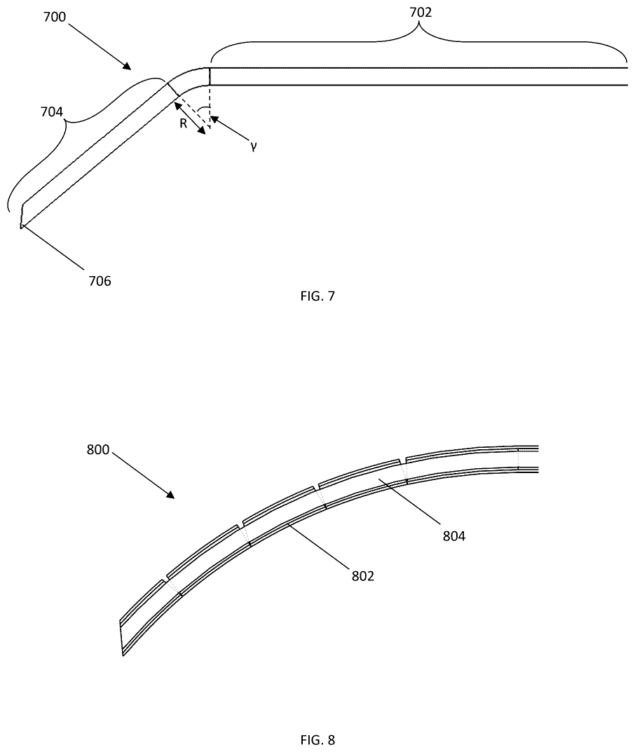

FIG. 7 is a schematic view drawing of an access needle 700 in a third example embodiment. The access needle 700 comprises a proximal portion 702 and a distal portion 704, both of which are substantially straight. The access needle 700 further comprises a tapered tip 706. The distal portion 704 may be bent with a bend angle γ at from about 30 to about 50 degrees with respect to the proximal portion 702, towards a lower surface of a deployer (i.e., in a direction that is facing the posterior direction of the eye). The bend radius, R may be 0.2 to 1 mm. The distal portion 704 of the access needle 700 may range in length from about 1.5 mm to about 2.5 mm. The distal portion 704 is pushed into a suprachoroidal space of an eye and is sufficiently long to serve as a guiding path for the implant to bend into the suprachoroidal space. At the same time, the distal portion 704 is not too long that it increases the risk of cutting through a choroid layer of the eye. As the implant moves over the access needle 700, the implant shields the tip 706 of the access needle 700, further lowering the risk of the access needle 700 causing unwanted trauma to the tissue.

In an alternative embodiment, the distal tip of the access needle follows a curved path, with a radius of curvature of from about 4 to about 6 mm. The tangent of the distal tip is at an angle of from about 30 to about 50 degrees relative to the horizontal of the needle.

When the access needle 700 is concentrically disposed within a guide tube (compare 400 of FIG. 4A), the diameter of the guide tube is within 0.6 mm larger than the diameter of the access needle 700, such that the access needle 700 is substantially straightened inside the guide tube. As the access needle 700 is extruded from the distal opening (compare 410 of FIG. 4A) of the guide tube, the guide tube acts to constrain the degree to which the access needle 700 can bend. Hence, the distal tip 706 of the access needle 700 forms a curved path as it is extruded, such that it traverses the ciliary body and dissects the suprachoroidal space of the eye.

The access needle 700 may be made of a flexible material with shape memory properties, such as Nitinol. As such, the access needle 700 retains its curvature despite passing through the straight ciliary sulcus guide. The stiffness of the access needle is no more than 2.5 N/mm, such that for an extruded length of 0.53 mm (length of the ciliary body), it can bend with an angle of more than 20 degrees with respect to its axis. If the access needle is too stiff, it will pierce through the sclera, rather than bending posteriorly into the suprachoroidal space. In one embodiment, an access needle made of superelastic Nitinol has a diameter of from about 0.13 mm to about 0.17 mm, such that this degree of stiffness is attained. To prevent buckling of the access needle, the free length of the access needle (i.e., it is not supported by any external clamp or tube) is kept to less than 8 mm.

The surface finish of the sharpened faces is no more than 4 microns (Ra), such that the tip can be sharp enough to cut through the ciliary body and smooth enough to slide along the inner surface of the choroid. The tapered or bevelled tip shapes may also facilitate atraumatic access through the ciliary body, such that bleeding is minimised. The surface finish of the tapered or bevelled tip should also be no more than 0.5 microns (RA), to minimise tissue trauma. This can be achieved by various surface treatments, e.g., electropolishing, black oxide surface treatment. Black oxide surface treatment may also help to prevent surface corrosion of the access needle.

FIG. 8 is a schematic view drawing of an access needle 800 in a fourth example embodiment. Flexibility of the access needle 800 is achieved by having flexible joints in the needle. This may be achieved by having multiple rigid segments e.g., 802 of the access needle 800, connected by a flexible internal core 804.

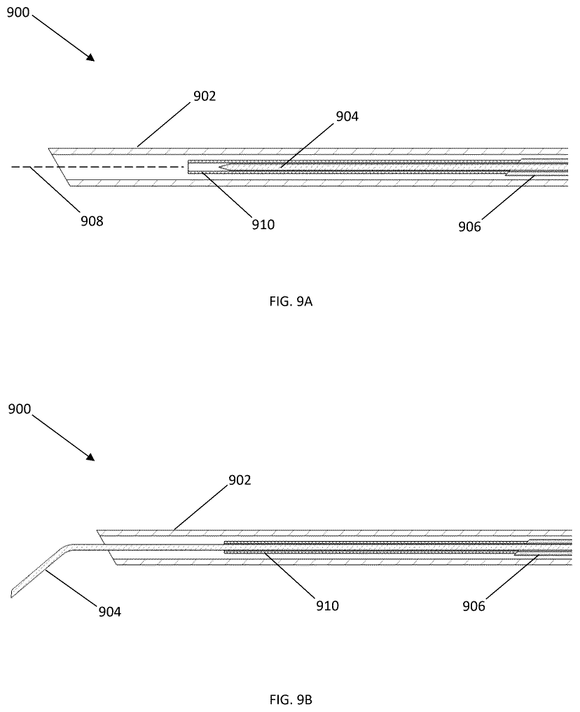

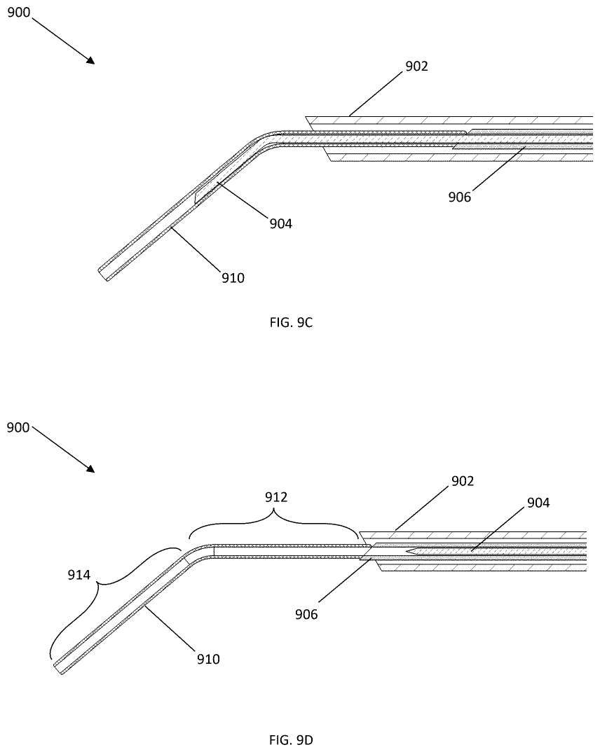

In a separate embodiment, the access needle is hollow. This permits the injection of fluid through the access needle. This can serve as a means of confirming whether the needle has indeed entered the potential space between the two layers. If it is in the right space, a loss of fluid resistance will be felt. This may be detected either in a tactile manner by an operator e.g., surgeon, or using a pressure sensor. FIG. 9A is a first schematic cross-sectional view of a portion of a deployer tool 900 in an example embodiment. The deployer tool 900 comprises a guide tube 902, an access needle 904 and an implant actuator 906 disposed within a lumen of the guide tube 902. The guide tube 902, access needle 904 and implant actuator 906 are arranged concentrically along a common longitudinal axis 908 such that the access needle 904 and implant actuator 906 can slide freely along the longitudinal axis 908.

An implant 910 in the form of an elongate hollow member is loaded on the access needle 904 such that a proximal end of the implant is engaged with a distal end of the implant actuator 906. The implant 910 has an inner diameter that is larger than the diameter of the access needle 904. The implant 910 is arranged such that it can slide freely along the access needle 904. The implant 910 is pre-formed with a bent configuration, straightened during deployment, and retains its bent configuration after deployment. The deployer tool 900 is used to deploy the implant 910 using an ab-interno approach from a sulcus or a portion of a ciliary body to a suprachoroidal space of the eye, such that when properly implanted, a proximal segment 912 is disposed in a posterior chamber of the eye and a distal segment 914 is disposed in the suprachoroidal space of the eye.

FIG. 9A shows the relative positions of the access needle 904, implant actuator 906, and implant 910 within the guide tube 902 when the guide tube 902 is abutted on the ciliary sulcus during an operation. The access needle 904, implant actuator 906, and implant 910 are in their retracted positions such that they are fully within the lumen of the guide tube 902.

FIG. 9B is a second schematic cross-sectional view of the portion of the deployer tool 900 in the example embodiment. FIG. 9B shows the relative positions of the access needle 904, implant actuator 906 and implant 910 when the access needle 904 is creating a delivery path during the operation.

During the creation of the delivery path, the access needle 904 is advanced in a distal direction such that it extends from a distal opening of the guide tube 902. The access needle 904 is advanced to cut through the ciliary body, contact an inner aspect of a scleral wall, bend and slide posteriorly along a curvature of the scleral wall, and dissect the suprachoroidal space. As the access needle 904 is pushed forward, the implant 910 is retained within the guide tube 902. The guide tube 902 remains abutted on the ciliary sulcus The implant 910 and implant actuator 906 remain in their retracted positions within the lumen of the guide tube 902.

FIG. 9C is a third schematic cross-sectional view of the portion of the deployer tool 900 in the example embodiment. FIG. 9C shows the relative positions of the access needle 904, implant actuator 906 and implant 910 when the implant actuator 906 is advancing the implant 910 along the delivery path created by the access needle 904 during the operation.

During insertion of the implant 910, the implant actuator 906 is advanced and pushes the implant 910 in a distal direction such that the implant 910 extends from the distal opening of the guide tube 902. The implant 910 slides over the access needle 904 along the delivery path to its intended position. The deployer 900 then extrudes the implant 910 such that its distal segment 914 is in the suprachoroidal space, whereas the proximal segment 912 remains in the posterior chamber.

FIG. 9D is a fourth schematic cross-sectional view of the portion of the deployer tool 900 in the example embodiment. FIG. 9D shows the relative positions of the access needle 904, implant actuator 906 and implant 910 after the access needle 904 is retracted into the guide tube 902 during the operation.

During retraction, the access needle 904 is retracted such that the distal tip of the access needle 904 is proximal relative to the distal tip of the implant actuator. Once the access needle 904 has no physical contact with the implant 910, the distal end of the implant actuator 906 is disengaged (i.e., break contact) from the proximal end of the implant 910. As the deployer 900 is retracted, it does not drag the implant 910 out with it.

In the example embodiment, the implant actuator 906 is a hollow tube. Its inner diameter can be from about 0.18 to about 0.25 mm, such that it is larger than the diameter of the access needle, and smaller than the outer diameter of the implant 910. In the example embodiment, the distal end/face of the implant actuator 906 abuts the proximal face of the implant 910. The cross-sectional profile of the distal tip of the implant actuator 906 can be a full circle; such that it has a hoop strength higher than that of the implant and does not get forced open by the implant 910. The distal tip of the implant actuator 906 is bevelled upwards, with a bevel angle of from about 40 to about 50 degrees, such that it is complementary to a bevelled tip shape on the proximal tip of the implant 910. This facilitates control of the rotational orientation of the implant.

In the example embodiment, the surface finish of the distal face of the implant actuator 906 may be no more than 0.5 microns (Ra), to ensure that the implant 910 does not get caught on it. The inner edge of the distal tip of the guide tube 902 may also be chamfered or radiused, to ensure that there are no burrs that catch the implant 910 as it is being released. The inner surface of the guide tube 902 may be smooth, with a surface finish of no more than 0.5 microns (Ra), such that it does not scratch or impede the sliding motion of the implant 910. This can be achieved by various types of treatment, such as chemical polishing, electropolishing etc.

In the example embodiment, after the implant 910 is pushed into position, there is preferably no contact with the deployer 900 on its inner and outer surfaces, such that it does not get dragged out with the deployer 900. The access needle 904 may be retracted fully into the implant actuator 906, such that its distal tip is a minimum of 0.2 mm proximal to the most proximal edge of the opening on the implant actuator 906. In other words, the distal tip of the access needle 904 is at least 0.2 mm to the left of the proximal edge of the implant actuator 906, as shown in FIG. 9D. The guide tube 902 may also be retracted relative to the implant actuator 906, such that its distal tip is a minimum of 0.2 mm proximal to the most proximal edge of the opening on the implant actuator 906. Throughout this process, the implant actuator 906 remains in position, i.e., it is at the same position relative to a housing of the deployer 900.

The guide tube 902 and access needle 904 may be withdrawn simultaneously for ease of use.

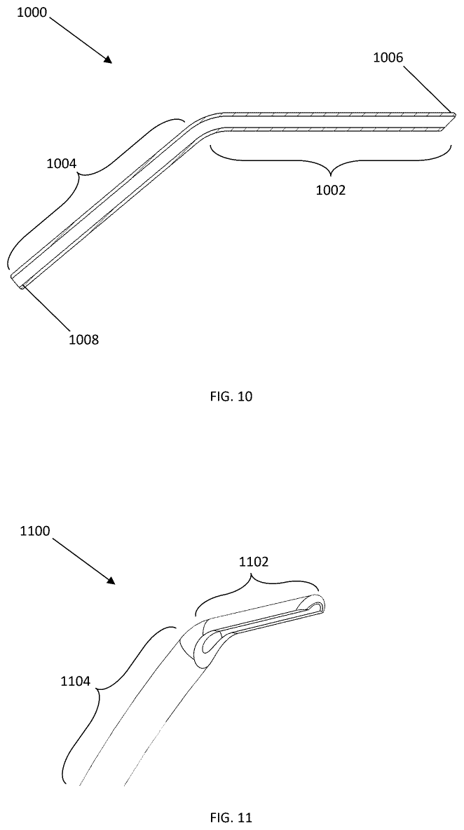

FIG. 10 is a schematic cross-sectional view drawing of an implant e.g., drainage shunt implant 1000 in a first example embodiment. The implant 1000 functions to conduct fluid e.g., aqueous humour from a posterior chamber to a suprachoroidal space of an eye/eyeball. The implant 1000 is in the form of a hollow elongate member/tubular member having a proximal segment 1002 configured to be disposed in the posterior chamber of the eye and a distal segment 1004 configured to be disposed in the suprachoroidal space of the eye. The implant 1000 further comprises a proximal tip 1006 in fluid communication with, and being configured to, receive fluid from the posterior chamber of the eye. The implant 1000 further comprises a distal tip 1008 in fluid communication with, and being configured to, discharge fluid into the suprachoroidal chamber of the eye.

The distal tip 1008 may be tapered to facilitate entry of the implant 1000; or blunted (radius<0.1 mm) to ensure it does not cause additional trauma e.g., cutting through the choroid tissue. As shown in FIG. 10, the distal tip 1008 is flat/blunted. The proximal tip 1006 of the implant 1000 is bevelled such that the bevel face is arranged to face away from a posterior aspect of an iris, when implanted in the eye.

The implant 1000 has an inner lumen with a diameter of from about 0.1 to about 0.2 mm. Combined with a total length of from about 6 to about 8 mm and a bent shape, the flow rate that can be achieved is from about 2 μL/min to about 20 μL/min, which is in the same order of magnitude as the physiological flow rate of aqueous in the eye. The implant 1000 may have a wall thickness of from about 0.05 to about 0.15 mm, such that the entry profile is sufficiently small, and the implant 1000 can still pass over an access needle, through ciliary tissue, into the suprachoroidal space.

The implant 1000 is configured to maintain a bent configuration that substantially complies with the anatomical curvature of the suprachoroidal space, when deployed in the eye, such that the proximal segment 1002 does not rub against an iris of the eye. This may be achieved in the following ways:

The implant 1000 may be pre-formed with a bent configuration, with a bend angle of from about 40 to about 60 degrees, and a bend radius of from about 0.5 mm to about 1 mm. During deployment, the implant 1000 may be straightened as it traverses the bent path before implantation. The implant 1004 may be sufficiently flexible that it retains its bent form despite this straightening (i.e., bending stress being less than yield strength). The implant 1000 may possess sufficient creep resistance that it retains its form despite being straightened for the duration of deployment (up to 10 minutes). The implant 1000 may be made of PTFE, with an inner diameter of 0.1 mm to 0.8 mm.

The implant 1000 or a part thereof may be sufficiently soft that it conforms to the anatomy of the eye (gets pressed down by the iris). The implant may be softer or almost as soft as the iris of the eye. Such a low Young's modulus may be achieved by a combination of one or more of the following: (1) a material that is very soft (Young's modulus<50 mPa), such as SIBS (2) a joint that permits flexing.

FIG. 11 is a perspective view drawing of an implant e.g., drainage shunt implant 1100 in a second example embodiment. The implant 1100 is in the form of a hollow elongate member having a proximal segment/section 1102 configured to be disposed in the posterior chamber of the eye and a distal segment/section 1104 configured to be disposed in the suprachoroidal space of the eye. The implant 1100 comprises cutouts at the proximal segment 1102 where a semi-cylindrical portion of material is removed, such that the proximal segment 1102 of the implant 1100 comprises a semi-cylindrical shape/profile. The cutouts reduce the cross-sectional area of the proximal segment, thereby reducing a second moment of area and hence lowering its bending strength.

FIG. 12 is a perspective view drawing of an implant e.g., drainage shunt implant 1200 in a third example embodiment. The implant 1200 is in the form of a hollow elongate member having a proximal segment/section 1202 configured to be disposed in the posterior chamber of the eye and a distal segment/section 1204 configured to be disposed in the suprachoroidal space of the eye. The implant 1200 comprises cutouts at a bent portion 1206 such that a part of the implant 1200 has a “spring” configuration in the form of a spiral 1206. This may, for example, be achieved by laser cutting a spiral in polyimide or nitinol material.

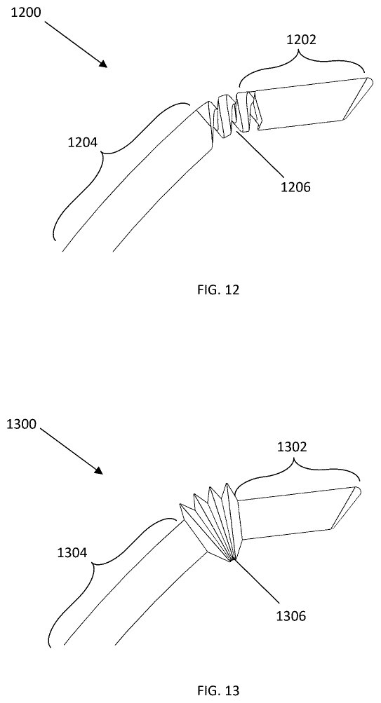

FIG. 13 is a perspective view drawing of an implant e.g., drainage shunt implant 1300 in a fourth example embodiment. The implant 1300 is in the form of a hollow elongate member having a proximal segment/section 1302 configured to be disposed in the posterior chamber of the eye and a distal segment/section 1304 configured to be disposed in the suprachoroidal space of the eye. The implant 1300 further comprises thin struts at a bend portion 1306 which confer a tubular shape while maintaining flexibility. These struts may, for example, take the shape of a coil, or modular rings connected by longitudinal segments. For example, as shown in FIG. 13, the bent portion 1306 has a “bellows” type configuration, with alternating rings of smaller and larger diameter formed of a rigid material such as Nitinol.

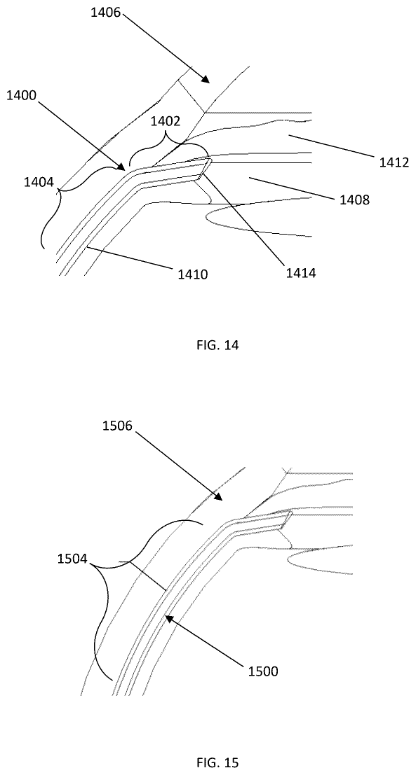

FIG. 14 is a schematic side view drawing of an implant e.g., drainage shunt implant 1400 inserted in an eye 1406 in an example embodiment. The implant 1400 is in the form of a hollow elongate member having a proximal segment/section 1402 configured to be disposed in a posterior chamber 1408 of the eye 1406 and a distal segment/section 1404 configured to be disposed in a suprachoroidal space 1410 of the eye 1406.

The lengths of the proximal segment 1402 and distal segment 1404 may be dimensioned to ensure a good fit.

For example, the proximal segment 1402 may have a length of from about 0.5 mm to about 1 mm, such that the proximal inlet is not occluded by surrounding tissue. For example, the length of the proximal segment may be from about 2 mm to about 4 mm such that it is sufficiently long to be seen when the iris 1412 is dilated. As shown in FIG. 14, the implant 1400 further comprises a proximal tip 1414 which is bevelled downwards at an angle (e.g., from about 30 to about 45 degrees), such that the likelihood of occlusion by the posterior iris 1412 is reduced. The edges of the proximal tip 1414 can be blunted (e.g., with a blunting radius of 0.05 mm or more) to avoid trauma to the iris 1412.

For example, the distal segment 1404 may have a length of from about 1 mm to about 3 mm. The distal segment 1404 of the implant 1400 may be straight or have a radius of curvature larger than its expected path within the suprachoroidal space (in a longitudinal or circumferential direction). This allows it to slide within the suprachoroidal space and prevents re-entry of the distal tip through the choroid layer into the vitreous cavity. FIG. 15 is a schematic side view drawing of an implant e.g., drainage shunt implant 1500 inserted in an eye 1506 in another example embodiment. As shown in FIG. 15, the implant 1500 has a distal segment 1504 that has a curved profile, with a radius of curvature that substantially mimics the radius of curvature of the sclera.

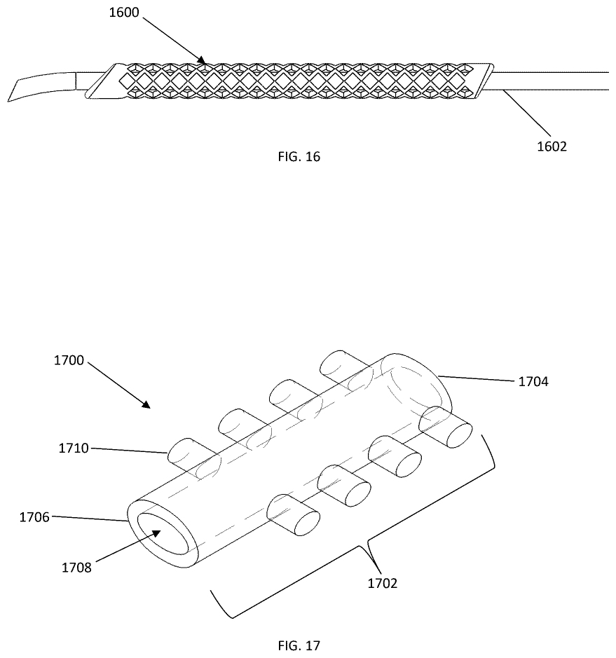

FIG. 16 is a side view drawing of an implant e.g., drainage shunt implant 1600 in a fifth example embodiment. The implant 1600 is made out of a wire structure (e.g., coil, mesh, slotted tube) that is able to expand radially in a deployed state. During insertion, the implant 1600 is maintained in its undeployed state, allowing it to coapt axially around an access needle 1602 with a relatively smaller outer diameter. A smaller difference in diameter between the access needle 1602 and implant 1600 facilitates greater ease of implant extrusion, as there is less tissue that the tip of the implant 1600 has to push apart. Once the implant 1600 is in the right position, it can be expanded radially to its optimal dimensions in its deployed state. This radial expansion could be achieved by, for example, having a balloon over the access needle 1602 that is inflated to deform the implant 1600, or making the implant 1600 from a shape memory material such as Nitinol and locking it in its undeployed state (e.g., by having a sheath over it). The implant is released after deployment.

FIG. 17 is a perspective view drawing of an implant e.g., drainage shunt implant 1700 in a sixth example embodiment. The implant 1700 is in the form of a hollow elongate member 1702 having a proximal end 1704, a distal end 1706, and a lumen 1708 extending between the proximal end 1704 and the distal end 1706. The proximal end 1704 is configured to be disposed in the posterior chamber of an eye and the distal end 1706 is configured to be disposed in a suprachoroidal space of the eye.

The implant 1700 further comprises a plurality of structural elements e.g., protrusions e.g., 1710 extending from an exterior surface of the hollow elongate member 1702. The plurality of protrusions e.g., 1710 are spaced apart along the hollow elongate member 1702 and are substantially perpendicular to a longitudinal axis of the hollow elongate member 1702. The plurality of protrusions e.g., 1710 function to provide a cross-sectional profile that maximises the surface area of the implant 1700 that is in contact with tissue in the suprachoroidal space. The plurality of protrusions e.g., 1710 also act as anchors such that the implant 1700 is immobilised in a substantially fixed position and does not substantially or easily migrate after implantation.

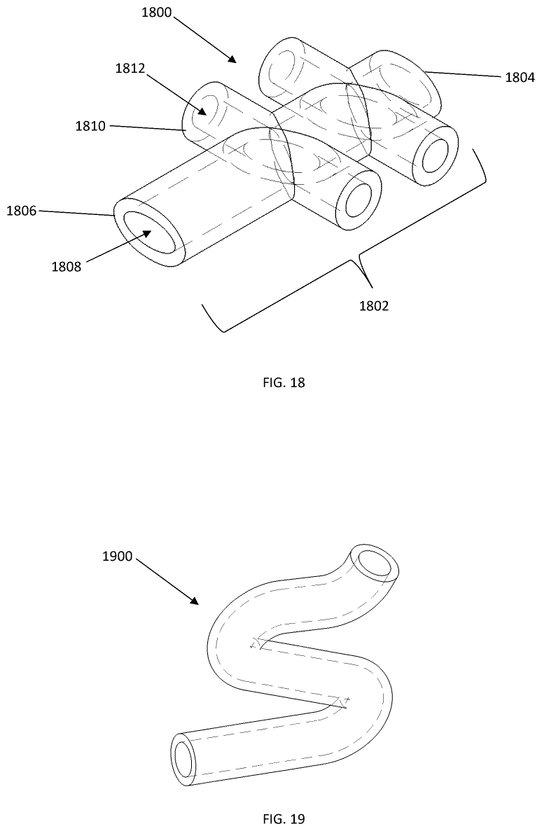

FIG. 18 is a perspective view drawing of an implant e.g., drainage shunt implant 1800 in a seventh example embodiment. The implant 1800 is in the form of a hollow elongate member 1802 having a proximal end 1804, a distal end 1806, and a lumen 1808 extending between the proximal end 1804 and the distal end 1806. The proximal end 1804 is configured to be disposed in the posterior chamber of an eye and the distal end 1806 is configured to be disposed in a suprachoroidal space of the eye.

The implant 1800 further comprises a plurality of structural elements e.g., protrusions e.g., 1810 extending from an exterior surface of the hollow elongate member 1802. Each protrusion 1810 comprises a lumen 1812 in fluid communication with the lumen 1808 of the hollow elongate member 1802. In the example embodiment, the plurality of protrusions e.g., 1810 comprises a plurality of lumens e.g., 1812 that are in fluid communication with the lumen 1808 of the hollow elongate member 1802, forming a network of interconnected lumens. The network of interconnected lumens may advantageously improve the conduction of fluid from the posterior chamber of the eye to the suprachoroidal space of the eye. The plurality of protrusions e.g., 1810 also function to provide a cross-sectional profile that maximises the surface area of the implant 1800 that is in contact with tissue in the suprachoroidal space. The plurality of protrusions e.g., 1810 also act as anchors such that the implant 1800 is immobilised in a substantially fixed position and does not substantially or easily migrate after implantation.

It will be appreciated that the plurality of protrusions e.g., 1710, 1810 are not limited to a cylindrical shape as illustrated in FIGS. 17 and 18. The plurality of protrusions e.g., 1710, 1810 may be in the form of other shapes, including but not limited to conical, pyramidal, hemispherical, and the like. It will also be appreciated that the plurality of protrusions e.g., 1710, 1810 are not limited to being arranged perpendicularly to the longitudinal axis of the hollow elongate member 1702, 1802 as illustrated in FIGS. 17 and 18. The plurality of protrusions e.g., 1710, 1810 may be formed at a slanted angle (e.g., between 30 to 60 degrees) with respect to the longitudinal axis of the hollow elongate member 1702, 1802. The plurality of protrusions e.g., 1710, 1810 may be slanted in the same direction (e.g., all the protrusions may be slanted towards the proximal end 1704, 1804 or all the protrusions may be slanted towards the distal end 1706, 1806) or in a mixture of directions (e.g., a group of protrusions may be slanted towards the proximal end 1704, 1804 and another group of protrusions may be slanted towards the distal end 1706, 1806).

FIG. 19 is a perspective view drawing of an implant e.g., drainage shunt implant 1900 in an eighth example embodiment. The implant 1900 is in the form of a hollow elongate member arranged in a curved configuration e.g., S-shaped configuration. The S-shaped configuration provides a cross-sectional profile that maximises the surface area of the implant 1800 that is in contact with tissue in the suprachoroidal space. The S-shaped configuration also facilitates immobilisation of the implant 1900 in a substantially fixed position such that the implant 1900 does not substantially or easily migrate after implantation.

### Applications

Embodiments of the disclosure provided herein provide an implant, deployer tool, system and method of creating and maintaining an outflow drainage of aqueous humour from the posterior chamber of the eye to the suprachoroidal space in patients with chronic ACG.

Advantageously, embodiments of the disclosed implant, deployer tool, system and method may provide a better improved approach to treat patients with chronic closed angle glaucoma with established peripheral anterior synechiae, in order to achieve sustained reductions in IOP while reducing hypotensive medication requirements. The placement of a drainage shunt in such a fashion in patients with chronic ACG allows augmentation of the unconventional outflow pathway, and drainage would not be compromised by future iris occlusion or reformation of PAS. Moreover, there is less risk of chronic and persistent endothelial cell loss due to the implant's position in the posterior chamber, compared with placement in the anterior chamber of the eye.

Even more advantageously, embodiments of the disclosed implant, deployer tool, system and method may be applied in various kinds of treatment for eye conditions, including for example: (i) treatment of chronic angle-closure glaucoma together with phaco-emulsification or clear lens extraction (i.e., concurrent placement of device in conjunction with lens extraction); (ii) treatment of chronic angle-closure glaucoma in pseudophakic patients (i.e., as a salvage treatment for patients with progressive disease despite previous lens extraction); and (iii) treatment of patients with glaucoma and concurrent corneal pathology (e.g., corneal dystrophies or post-keratoplasty) leading to a relative contraindication for conventional anterior chamber-placed MIGS devices

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

1. An implant configured to be deployed in an eye by a deployer tool to facilitate conduction of fluid from a posterior chamber to a suprachoroidal space of the eye, the implant comprising,
an elongate member having a proximal segment configured to be disposed in the posterior chamber of the eye and a distal segment configured to be disposed in the suprachoroidal space of the eye,
wherein the elongate member is configured to maintain a bent configuration that substantially complies with anatomical curvatures of the posterior chamber and the suprachoroidal space, when deployed in the eye.

2. The implant according to claim 1, wherein the elongate member is configured to be straightened to facilitate loading of the implant into the deployer tool for deployment; and to return to the bent configuration when deployed in the eye.

3. The implant according to claim 1, wherein the elongate member is pre-formed in the bent configuration with a bend angle that substantially matches an anatomical angle located between the posterior chamber and the suprachoroidal space of the eye.

4. The implant according to claim 1, wherein the elongate member has a bending stiffness of no more than 0.5 N.mm², such that the elongate member is able to move along a delivery path formed by an access needle of the deployer tool from a sulcus or a portion of the ciliary body to the suprachoroidal space of the eye, without substantially deforming the access needle.

5. The implant according to claim 1, wherein the distal segment of the elongate member is curved with a radius of curvature that is substantially identical to a radius of curvature of an inner sclera of the eye.

6. The implant according to claim 1, wherein the elongate member is bevelled at its proximal tip such that the bevelled surface is configured to face away from a posterior surface of an iris, when the implant is deployed in the eye.

7. A deployer tool for inserting an implant into an eye to facilitate conduction of fluid from a posterior chamber to a suprachoroidal space of the eye, the deployer tool comprising,
a guide tube for accessing the posterior chamber of the eye through an insertion site, said guide tube comprising a distal tip that is configured to abut a ciliary sulcus or a portion of a ciliary body of the eye; and
an access needle configured to be disposed within the guide tube and to create a delivery path from the sulcus or a portion of the ciliary body to the suprachoroidal space of the eye;
wherein the deployer tool allows for movement of the implant in relation to the access needle along the delivery path such that a distal segment of the implant is disposed in the suprachoroidal space of the eye, and a proximal segment of the implant is disposed in the posterior chamber of the eye.

8. The deployer tool according to claim 7, further comprising an implant actuator configured to detachably engage to the implant and configured to move the implant distally in relation to the access needle along the delivery path.

9. The deployer tool according to claim 7,
wherein the access needle comprises a distal tip configured to dissect tissue so as to create the delivery path;
wherein the delivery path comprises a first section extending from the sulcus or a portion of the ciliary body to an inner surface of a sclera and a second section extending from the inner surface of the sclera to the suprachoroidal space; and
wherein the first section and second section form a bent angle along the delivery path.

10. The deployer tool according to claim 9, wherein the access needle is pre-formed in a bent configuration and configured to be straightened when disposed within the guide tube and to return to the bent configuration when extended from the distal tip of the guide tube for the creation of the delivery path.

11. The deployer tool according to claim 9, wherein the access needle has a stiffness of no more than 2.5 N/mm to facilitate bending of the access needle at the bent angle along the delivery path.

12. The deployer tool according to claim 7, wherein the guide tube has a Young's modulus of at least 7.5 GPa such that the guide tube does not substantially deflect when abutting the ciliary sulcus or a portion of the ciliary body of the eye.

13. The deployer tool according to claim 8, further comprising an access needle holder, an implant actuator holder and a guide tube holder disposed thereon,
wherein the access needle holder is configured to engage a proximal end of the access needle, the implant actuator holder is configured to engage the implant actuator and the guide tube holder is configured to engage the guide tube, and
optionally wherein the access needle holder, implant actuator holder and/or the guide tube holder are each further coupled to a respective control configured to actuate the access needle, implant actuator and optionally, the guide tube.

14. A system for facilitating the conduction of fluid from a posterior chamber to a suprachoroidal space of an eye, the system comprising,
an implant comprising,
an elongate member having a proximal segment and a distal segment,
wherein the elongate member is configured to maintain a bent configuration that substantially complies with anatomical curvatures of the posterior chamber and the suprachoroidal space, when deployed in the eye; and
a deployer tool comprising,
a guide tube for accessing the posterior chamber of the eye through an insertion site, said guide tube comprising a distal tip that is configured to abut a ciliary sulcus or a portion of a ciliary body of the eye; and
an access needle configured to be disposed within the guide tube and to create a delivery path from the sulcus or a portion of the ciliary body to the suprachoroidal space of the eye;
wherein the deployer tool allows for movement of the implant in relation to the access needle in a distal direction along the delivery path such that a distal segment of the implant is disposed in the suprachoroidal space of the eye, and a proximal segment of the implant is disposed in the posterior chamber of the eye.

15. The system according to claim 14, wherein the elongate member is configured to be straightened to facilitate loading of the implant on a distal end of the access needle.

16. The system according to claim 15, wherein the implant and access needle are configured to be in a retracted position and disposed within the guide tube prior to deployment.

17. The system according to claim 16,
wherein the insertion site is located on a cornea of the eye;
wherein the distal tip of the guide tube is configured to be inserted through the corneal insertion site into an anterior chamber of the eye, and to be inserted across a pupil of the eye, and to be moved along a posterior surface of an iris to abut the ciliary sulcus or a portion of the ciliary body of the eye.

18. The system according to claim 17, wherein the deployer tool further comprises,
an access needle holder configured to engage a proximal end of the access needle and configured to extend the access needle distally from the distal tip of the guide tube to allow a distal tip of the access needle to dissect tissue and create the delivery path;
wherein the delivery path comprises a first section extending from the sulcus or a portion of the ciliary body to an inner surface of a sclera and a second section extending from the inner surface of the sclera to the suprachoroidal space; and
wherein the first section and second section form a bent angle along the delivery path.

19. The system according to claim 18, wherein the deployer tool further comprises,
an implant actuator configured to detachably engage the implant; and
an implant actuator holder configured to engage the implant actuator and configured to move the implant actuator such that the implant actuator engages the implant and the implant is moved distally in relation to the access needle along the delivery path.

20. The system according to claim 18, wherein the access needle holder is further configured to retract the access needle proximally such that the distal tip of the access needle is more proximal in relation to the distal tip of the implant actuator.
